SEQUENCE LISTING (15 sequences)

SEQ ID NO: 1 - DNA, 1788 bp, Populus nigra
  CDS (1)..(1788)
atggcaactgaattattgtgcttgcaccgtccaatctcactgacacacaaattgttcagaaatcccttgcctaaagtcatccaggccactcccttaactttgaaactcagatgttctgtaagcacagaaaacgtcagcttcacagaaacagaaacagaaaccagaaggtctgccaattatgaaccaaatagctgggattatgattatttgctgtcttcggacactgacgaatcgattgaagtatacaaagacaaggccaaaaagctggaggctgaggtgagaagagagattaacaatgaaaaggcagagtttttgactctgcctgaactgatagataatgtccaaaggttaggattaggttaccggttcgagagtgacataaggagagcccttgatagatttgtttcttcaggaggatttgatgctgttacaaaaactagccttcatgctactgctcttagcttcaggcttctcagacagcatggctttgaggtctctcaagaagcgttcagcggattcaaggatcaaaatggcaatttcttgaaaaaccttaaggaggacatcaaggcaatactaagcctatatgaagcttcatttcttgccttagaaggagaaaatatcttggatgaggccaaggtgtttgcaatatcacatctaaaagagctcagcgaagaaaagattggaaaagacctggccgaacaggtgaatcatgcattggagcttccattgcatcgaaggacgcaaagactagaagctgtttggagcattgaagcataccgtaaaaaggaagatgcagatcaagtactgctagaacttgctatattggactacaacatgattcaatcagtataccaaagagatcttcgcgagacatcaaggtggtggaggcgtgtgggtcttgcaacaaagttgcattttgctagagacaggttaattgaaagcttttactgggcagttggagttgcgtttgaacctcaatacagtgattgccgtaattccgtagcaaaaatgttttcgtttgtaacaatcattgatgatatctatgatgtttatggtactctggatgagttggagctatttacagatgctgttgagagatgggatgttaatgccatcgatgatcttccggattatatgaagctctgcttcctagctctctataacactatcaatgagatagcttatgataatctgaaggacaagggggaaaacattcttccatacctaacaaaagcgtgggcagatttatgcaatgcattcctacaagaagcaaaatggttgtacaataagtccacaccaacatttgatgaatatttcggaaatgcatggaaatcatcctcagggcctcttcaactagtttttgcctactttgccgttgttcaaaacatcaagaaagaggaaattgataacttacaaaagtatcatgatatcatcagtaggccttcccacatctttcgtctttgcaacgacttggcttcagcatcggctgagatagcgagaggtgaaaccgcgaattctgtatcatgctacatgcgtacaaaaggcatttctgaggaacttgctactgaatccgtaatgaatttgatcgacgaaacctggaaaaagatgaacaaagaaaagcttggtggctctctgtttgcaaaaccttttgtcgaaacagctattaaccttgcacgacaatcccattgcacttatcacaacggagatgcgcatacttcaccagatgagctcactaggaaacgtgtcctgtcagtaatcacagagcctattctaccctttgagagataa

SEQ ID NO: 2 - protein, 595 aa, Populus nigra
MATELLCLHRPISLTHKLFRNPLPKVIQATPLTLKLRCSVSTENVSFTETETETRRSANYEPNSWDYDYLLSSDTDESIEVYKDKAKKLEAEVRREINNEKAEFLTLPELIDNVQRLGLGYRFESDIRRALDRFVSSGGFDAVTKTSLHATALSFRLLRQHGFEVSQEAFSGFKDQNGNFLKNLKEDIKAILSLYEASFLALEGENILDEAKVFAISHLKELSEEKIGKDLAEQVNHALELPLHRRTQRLEAVWSIEAYRKKEDADQVLLELAILDYNMIQSVYQRDLRETSRWWRRVGLATKLHFARDRLIESFYWAVGVAFEPQYSDCRNSVAKMFSFVTIIDDIYDVYGTLDELELFTDAVERWDVNAIDDLPDYMKLCFLALYNTINEIAYDNLKDKGENILPYLTKAWADLCNAFLQEAKWLYNKSTPTFDEYFGNAWKSSSGPLQLVFAYFAVVQNIKKEEIDNLQKYHDIISRPSHIFRLCNDLASASAEIARGETANSVSCYMRTKGISEELATESVMNLIDETWKKMNKEKLGGSLFAKPFVETAINLARQSHCTYHNGDAHTSPDELTRKRVLSVITEPILPFER

SEQ ID NO: 3 - DNA, 1269 bp, Arabidopsis thaliana
  CDS (1)..(1269)
atgttattcacgaggagtgttgctcggatttcttctaagtttctgagaaaccgtagcttctatggctcctctcaatctctcgcctctcatcggttcgcaatcattcccgatcagggtcactcttgttctgactctccacacaagggttacgtttgcagaacaacttattcattgaaatctccggtttttggtggatttagtcatcaactctatcaccagagtagctccttggttgaggaggagcttgacccattttcgcttgttgccgatgagctgtcacttcttagtaataagttgagagagatggtacttgccgaggttccaaagcttgcctctgctgctgagtacttcttcaaaaggggtgtgcaaggaaaacagtttcgttcaactattttgctgctgatggcgacagctctggatgtacgagttccagaagcattgattggggaatcaacagatatagtcacatcagaattacgcgtaaggcaacggggtattgctgaaatcactgaaatgatacacgtcgcaagtctactgcacgatgatgtcttggatgatgccgatacaaggcgtggtgttggttccttaaatgttgtaatgggtaacaagatgtcggtattagcaggagacttcttgctctcccgggcttgtggggctctcgctgctttaaagaacacagaggttgtagcattacttgcaactgctgtagaacatcttgttaccggtgaaaccatggagataactagttcaaccgagcagcgttatagtatggactactacatgcagaagacatattataagacagcatcgctaatctctaacagctgcaaagctgttgccgttctcactggacaaacagcagaagttgccgtgttagcttttgagtatgggaggaatctgggtttagcattccaattaatagacgacattcttgatttcacgggcacatctgcctctctcggaaagggatcgttgtcagatattcgccatggagtcataacagccccaatcctctttgccatggaagagtttcctcaactacgcgaagttgttgatcaagttgaaaaagatcctaggaatgttgacattgctttagagtatcttgggaagagcaagggaatacagagggcaagagaattagccatggaacatgcgaatctagcagcagctgcaatcgggtctctacctgaaacagacaatgaagatgtcaaaagatcgaggcgggcacttattgacttgacccatagagtcatcaccagaaacaagtga

SEQ ID NO: 4 - protein, 422 aa, Arabidopsis thaliana
MLFTRSVARISSKFLRNRSFYGSSQSLASHRFAIIPDQGHSCSDSPHKGYVCRTTYSLKSPVFGGFSHQLYHQSSSLVEEELDPFSLVADELSLLSNKLREMVLAEVPKLASAAEYFFKRGVQGKQFRSTILLLMATALDVRVPEALIGESTDIVTSELRVRQRGIAEITEMIHVASLLHDDVLDDADTRRGVGSLNVVMGNKMSVLAGDFLLSRACGALAALKNTEVVALLATAVEHLVTGETMEITSSTEQRYSMDYYMQKTYYKTASLISNSCKAVAVLTGQTAEVAVLAFEYGRNLGLAFQLIDDILDFTGTSASLGKGSLSDIRHGVITAPILFAMEEFPQLREVVDQVEKDPRNVDIALEYLGKSKGIQRARELAMEHANLAAAAIGSLPETDNEDVKRSRRALIDLTHRVITRNK

SEQ ID NO: 5 - DNA, 912 bp, Solanum lycopersicum
  CDS (1)..(912)
atgagttctttggttcttcaatgttggaaattatcatctccatctctgattttacaacaaaatacatcaatatccatgggtgcattcaaaggtattcataaacttcaaatcccaaattcgcctctgacagtgtctgctcgtggactcaacaagatttcatgctcactcaacttacaaaccgaaaagctttgttatgaggataatgataatgatcttgatgaagaacttatgcctaaacacattgctttgataatggatggtaataggagatgggcaaaggataagggtttagaagtatatgaaggtcacaaacatattattccaaaattaaaagagatttgtgacatttcttctaaattgggaatacaaattatcactgcttttgcattctctactgaaaattggaaacgatccaaggaggaggttgatttcttgttgcaaatgttcgaagaaatctatgatgagttttcgaggtctggagtaagagtgtctattataggttgtaaatccgacctcccaatgacattacaaaaatgcatagcattaacagaagagactacaaagggcaacaaaggacttcaccttgtgattgcactaaactatggtggatattatgacatattgcaagcaacaaaaagcattgttaataaagcaatgaatggtttattagatgtagaagatatcaacaagaatttatttgatcaagaacttgaaagcaagtgtccaaatcctgatttacttataaggacaggaggtgaacaaagagttagtaactttttgttgtggcaattggcttacactgaattttacttcaccaacacattgtttcctgattttggagaggaagatcttaaagaggcaataatgaactttcaacaaaggcatagacgttttggtggacacacatattga

SEQ ID NO: 6 - protein, 303 aa, Solanum lycopersicum
MSSLVLQCWKLSSPSLILQQNTSISMGAFKGIHKLQIPNSPLTVSARGLNKISCSLNLQTEKLCYEDNDNDLDEELMPKHIALIMDGNRRWAKDKGLEVYEGHKHIIPKLKEICDISSKLGIQIITAFAFSTENWKRSKEEVDFLLQMFEEIYDEFSRSGVRVSIIGCKSDLPMTLQKCIALTEETTKGNKGLHLVIALNYGGYYDILQATKSIVNKAMNGLLDVEDINKNLFDQELESKCPNPDLLIRTGGEQRVSNFLLWQLAYTEFYFTNTLFPDFGEEDLKEAIMNFQQRHRRFGGHTY

SEQ ID NO: 7 - DNA, 2337 bp, Solanum lycopersicum
  CDS (1)..(2337)
atgatagttggctatagaagcacaatcataaccctttctcatcctaagctaggcaatgggaaaacaatttcatccaatgcaattttccagagatcatgtagagtaagatgcagccacagtaccacttcatcaatgaatggtttcgaagatgcaagggatagaataagggaaagttttgggaaattagagttatctccttcttcctatgacacagcatgggtagctatggtcccttcaagacattcactaaatgagccatgttttccacaatgtttggattggattattgaaaatcaaagagaagatggatcttggggactaaaccctacccatccattgcttctaaaggactcactttcttccactcttgcatgtttgcttgcactaaccaaatggagagttggagatgagcaaatcaaaagaggtcttggcttcattgaaacgtatggttgggcagtagataacaaggatcaaatttcacctttaggatttgaagttatattttctagtatgatcaaatctgcagagaaattagatttaaatttgcctttgaatcttcatcttgtaaatttggtgaaatgcaaaagagattcaacaattaaaaggaatgttgaatatatgggtgaaggagttggtgaattatgtgattggaaggaaatgataaagttacatcaaagacaaaatggttcattatttgattcaccagccactactgcagctgccttgatttatcatcaacatgatcaaaaatgctatcaatatcttaattcaatcttccaacaacacaaaaattgggttcccactatgtatccaacaaaggtacattcattgctttgcttggttgatacacttcaaaatcttggagtacatcggcattttaaatcagaaataaagaaagctctagatgaaatatacaggctatggcaacaaaagaatgaacaaattttctcaaatgtcacccattgtgctatggcttttcgacttctaaggatgagctactatgatgtctcctcagatgaactagcagaatttgtggatgaagaacatttctttgcaacaaatgggaaatataaaagtcatgttgaaattcttgaactccacaaagcatcacaattggctattgatcatgagaaagatgacattttggataaaataaacaattggacaagagcttttatggagcaaaaactcttaaacaatggcttcatagataggatgtcaaagaaagaggtggaacttgctttgaggaagttttataccacatctcatctagcagaaaatagaagatatataaagtcatacgaagagaacaattttaaaatcttaaaagcagcttataggtcacccaacattaacaataaggacttgttagcattttcaatacacgactttgaattatgccaagctcaacaccgagaagaacttcaacaactcaagaggtggtttgaagattatagattggaccaactcggacttgcagaacgatatatacatgctagttacttatttggtgttactgttatccccgagcctgaattatccgatgctcgcctcatgtacgcgaaatacgtcatgctcctgactattgtcgatgatcatttcgagagttttgcatctaaagatgaatgtttcaacatcattgaattagtagaaaggtgggatgactatgcaagtgtaggttataaatctgagaaggttaaagtttttttttctgttttctataaatcaatagaggagcttgcaacaattgctgaaattaaacaaggacgatccgtcaaaaatcaccttattaatttgtggcttgaattgatgaagttgatgttgatggagcgagtagagtggtgttctggcaagacaataccaagcatagaagagtacttgtatgttacatctataacattttgtgcaaaattgattcctctctcaacacaatattttcttggaataaaaatatccaaagatctactagaaagtgatgaaatatgtggcctatggaattgtagcggtagagtgatgcgaatccttaatgatttacaagattccaagagagaacaaaaggaggtctcaataaatttagtcacattactaatgaaaagtatgtctgaggaagaagctataatgaagataaaggaaatcttggaaatgaatagaagagagttattgaaaatggttttagttcaaaaaaagggaagccaattgcctcaattatgcaaagatatattttggaggacaagcaaatgggctcatttcacttattcacaaactgatggatatagaattgcagaggaaatgaagaatcacattgatgaagtcttttacaaaccactcaatcattaa

SEQ ID NO: 8 - protein, 778 aa, Solanum lycopersicum
MIVGYRSTIITLSHPKLGNGKTISSNAIFQRSCRVRCSHSTTSSMNGFEDARDRIRESFGKLELSPSSYDTAWVAMVPSRHSLNEPCFPQCLDWIIENQREDGSWGLNPTHPLLLKDSLSSTLACLLALTKWRVGDEQIKRGLGFIETYGWAVDNKDQISPLGFEVIFSSMIKSAEKLDLNLPLNLHLVNLVKCKRDSTIKRNVEYMGEGVGELCDWKEMIKLHQRQNGSLFDSPATTAAALIYHQHDQKCYQYLNSIFQQHKNWVPTMYPTKVHSLLCLVDTLQNLGVHRHFKSEIKKALDEIYRLWQQKNEQIFSNVTHCAMAFRLLRMSYYDVSSDELAEFVDEEHFFATNGKYKSHVEILELHKASQLAIDHEKDDILDKINNWTRAFMEQKLLNNGFIDRMSKKEVELALRKFYTTSHLAENRRYIKSYEENNFKILKAAYRSPNINNKDLLAFSIHDFELCQAQHREELQQLKRWFEDYRLDQLGLAERYIHASYLFGVTVIPEPELSDARLMYAKYVMLLTIVDDHFESFASKDECFNIIELVERWDDYASVGYKSEKVKVFFSVFYKSIEELATIAEIKQGRSVKNHLINLWLELMKLMLMERVEWCSGKTIPSIEEYLYVTSITFCAKLIPLSTQYFLGIKISKDLLESDEICGLWNCSGRVMRILNDLQDSKREQKEVSINLVTLLMKSMSEEEAIMKIKEILEMNRRELLKMVLVQKKGSQLPQLCKDIFWRTSKWAHFTYSQTDGYRIAEEMKNHIDEVFYKPLNH

SEQ ID NO: 9 - DNA, 1746 bp, Lavandula angustifolia
  CDS (1)..(1746)
atgtctaccattattgcgatacaagtgttgcttcctattccaactactaaaacataccctagtcatgacttggagaagtcctcttcgcggtgtcgctcctcctccactcctcgccctagactgtgttgctcgttgcaggtgagtgatccgatcccaacgggccggcgatccggaggctacccgcccgccctatgggatttcgacactattcaatcgctcaacaccgagtataagggagagaggcacatgagaagggaagaagacctaattgggcaagttagagagatgctggtgcatgaagtagaggatcccactccacagctggagttcattgatgatttgcataagcttggcatatcttgccattttgagaatgaaatcctccaaatcttgaaatccatatatcttaatcaaaactacaaaagggatttgtactcaacatctctagcattcagactcctcagacaatatggcttcatccttccacaagaagtatttgattgtttcaagaatgaggagggtacggatttcaagccaagcttcggccgtgatatcaaaggcttgttacaattgtatgaagcttctttcctatcaagaaaaggagaagaaactttacaactagcaagagagtttgcaacaaagattctgcaaaaagaagttgatgagagagagtttgcaaccaagatggagttcccttctcattggacggttcaaatgccgaatgcaagacctttcatcgatgcttaccgtaggaggccggatatgaatccagttgtgctcgagctagccatacttgatacaaatatagttcaagcacaatttcaagaagaactcaaagagacctcaaggtggtgggagagtacaggcattgtccaagagcttccatttgtgagggataggattgtggaaggctacttttggacgattggagtgactcagagacgcgagcatggatacgaaagaatcatgaccgcaaaggttattgccttagtaacatgtttagacgacatatacgatgtttatggcacgatagaagagcttcaacttttcacaagcacaatccaaagatgggatttggaatcaatgaagcaactccctacctacatgcaagtaagctttcttgcactacacaactttgtaaccgaggtggcttacgatactctcaagaaaaagggctacaactccacaccatatttaagaaaaacgtgggtggatcttgttgaatcatatatcaaagaggcaacttggtactacaacggttataaacctagtatgcaagaataccttaacaatgcatggatatcagtcggaagtatggctatactcaaccacctcttcttccggttcacaaacgagagaatgcataaataccgcgatatgaaccgtgtctcgtccaacattgtgaggcttgctgatgatatgggaacatcattggctgaggtggagagaggggacgtgccgaaagcaattcaatgctacatgaatgagacgaatgcttctgaagaagaagcaagagaatatgtaagaagagtcatacaggaagaatgggaaaagttgaacacagaattgatgcgggatgatgatgatgatgatgattttacactatccaaatattactgtgaggtggttgctaatcttacaagaatggcacagtttatataccaagatggatcggatggcttcggcatgaaagattccaaggttaatagactgctaaaagagacgttgatcgagcgctacgaataa

SEQ ID NO: 10 - protein, 581 aa, Lavandula angustifolia
MSTIIAIQVLLPIPTTKTYPSHDLEKSSSRCRSSSTPRPRLCCSLQVSDPIPTGRRSGGYPPALWDFDTIQSLNTEYKGERHMRREEDLIGQVREMLVHEVEDPTPQLEFIDDLHKLGISCHFENEILQILKSIYLNQNYKRDLYSTSLAFRLLRQYGFILPQEVFDCFKNEEGTDFKPSFGRDIKGLLQLYEASFLSRKGEETLQLAREFATKILQKEVDEREFATKMEFPSHWTVQMPNARPFIDAYRRRPDMNPVVLELAILDTNIVQAQFQEELKETSRWWESTGIVQELPFVRDRIVEGYFWTIGVTQRREHGYERIMTAKVIALVTCLDDIYDVYGTIEELQLFTSTIQRWDLESMKQLPTYMQVSFLALHNFVTEVAYDTLKKKGYNSTPYLRKTWVDLVESYIKEATWYYNGYKPSMQEYLNNAWISVGSMAILNHLFFRFTNERMHKYRDMNRVSSNIVRLADDMGTSLAEVERGDVPKAIQCYMNETNASEEEAREYVRRVIQEEWEKLNTELMRDDDDDDDFTLSKYYCEVVANLTRMAQFIYQDGSDGFGMKDSKVNRLLKETLIERYE

SEQ ID NO: 11 - DNA, 5803 bp, Artificial
  Gene cluster
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggagatcggtacttcgcgaatgcgtcgagatggcgcgccttggtaaaggatatatggtagtatttgcaggggatacatagggagatatagaagttctaccaataggttcagaatttagccatagttctgatctaaaagctgtactagacgtatttgatactgtaaaggtatttatatataacttaactcctgaatcctctgggtttatcaagcctccccaagcatttaaattacgttctagaattagaaagggggtatgacccataaaatattttcctttaaaagattcatatacacgatagggtaggtttactactttagttggtttatcatgatgacttacggtatcggaatttaaaatgttttgattttccataaatatgacctcctagtatttagtattattttatgtaaatatatatgtagaagtgtaccatttgtgcaagatttcaataaagggtatattttacctatttttttagtataaaaaatgcaaaaaatatgaacaaaagtagagttcctatgtattaaattgtaaaatatccactaaaaaaataaaattataataaaaaatacaaaaaaataattgacaatatataaataattatgcataattatatcatgataacaattagttaagcataattacatatatatgaacataatatgacatcttagaagcatatctttcgttagtaataatataatttcctttagaagaaaatgatttatttaaaataaatagtgtaatgttttttataatttcaaaaagttccccaatttagcatactaggcatgataaaaatagcttgaataagtgcccgggattatttattgatacatagagaatttcactctttgcattttatctaacatcaaggggtttatttgtcacaaattatgtaaaaataaaacaaagatgtaagaaagtcctatgatataaattttgtaaacataataaattagctttcataagattggaagaatgataattactacttagaactgctaaaaattaggaaagaggtgtcgttaattataccgttcgtataatgtatgctatacgaagttatttcagattaaatttttgcttatttgatttacattatataatattgagtaaagtattgactagcaaaattttttgatactttaatttgtgaaatttcttatcaaaagttatatttttgaatgatttttattgaaaaatacaactaaaaaggattatagtataagtgtgtgtaattttgtgttaaatttaaagggaggaaatgaacatgaactttaataaaattgatttagacaattggaagagaaaagagatatttaatcattatttgaaccaacaaacgacttttagtataaccacagaaattgatattagtgttttatacagaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatttaaaagcaaatataaatgaaaaattgaaccctagcattatgtaaatgcagggtttaatttttatattaagcagcataatagaaagttttttaaatgcatgtatatatggggtatttaaagggaaatctataatataatttaggactatataacttcgtataatgtatgctatacgaacggtacctaggatatataataaattgaatatagtaaacaaaaagggacatatttataatatgttctttttagtttaatactcaatttttgcacataagaaattaacttaatataaaaaaatttgcgaagctttgcttcgcagtttaatattgtttaggtggttaaattatgaatctggaagtgttaaaaacagagtttaagtatttaagagataaaataattgaaaagcaatatgaacatcttgatcctatgcaaagaaaagcagttttaaatggtgaaaataactgtattgttattgcttgtcctggagcaggaaagacccagactattattaatagagtggactacttatgtagattcggtcctatatacaatacagattatgtacctaattgtctaaagaccgatgatttacagataatgaagaaatatttaaatgataattcttttaaagatgtgactgcagtaaataaaattgagcatttgttaaatagcaataaaataaatccacagaacatagttgttataacttttactagagcagctgctctcaatatgaaaaacagatacatatctataggaaataaagaaaagtcacctttttttggaacattccactccctattttataatatattgaaaaagcataataaagaaataaatattatagatccttataaggcacatgagatagttaaaaatacacttatgtattatctggactttataggagaagagagagtaaaggaagttctaaatgacatatctcttttaaaaaatagtgaaactaacatagatttatttaaaagtaaaattgacaaaagtgtatttttaaaatgttttaatgaatatgaaaattataaagctagaaataagcttatggattttgatgatttacaattaaaagttaaagatatgtttctaaatcagaaatctattctagatagttatcagaatttgttcaagtatattttagttgatgagtttcaggattcagataacctccaaatattcgaaatcggatgccgggaccgacgagtgcagaggcgtgcaagcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 12 - DNA, 10704 bp, Artificial
  Gene cluster
gaattcgagctcggtacccggggatcctctagagtcgacctgcaggcatgcccgcggtcgactttttaacaaaatatattgataaaaataataatagtgggtataattaagttgttagagaaaacgtataaattagggataaactatggaacttatgaaatagattgaaatggtttatctgttaccccgtaggatccagaatttaaaaggagggattaaacatatgaatggtttcgaagatgcaagggatagaataagggaaagttttgggaaattagagttatctccttcttcctatgacacagcatgggtagctatggtcccttcaagacattcactaaatgagccatgttttccacaatgtttggattggattattgaaaatcaaagagaagatggatcttggggactaaaccctacccatccattgcttctaaaggactcactttcttccactcttgcatgtttgcttgcactaaccaaatggagagttggagatgagcaaatcaaaagaggtcttggcttcattgaaacgtatggttgggcagtagataacaaggatcaaatttcacctttaggatttgaagttatattttctagtatgatcaaatctgcagagaaattagatttaaatttgcctttgaatcttcatcttgtaaatttggtgaaatgcaaaagagattcaacaattaaaaggaatgttgaatatatgggtgaaggagttggtgaattatgtgattggaaggaaatgataaagttacatcaaagacaaaatggttcattatttgattcaccagccactactgcagctgccttgatttatcatcaacatgatcaaaaatgctatcaatatcttaattcaatcttccaacaacacaaaaattgggttcccactatgtatccaacaaaggtacattcattgctttgcttggttgatacacttcaaaatcttggagtacataggcattttaaatcagaaataaagaaagctctagatgaaatatacaggctatggcaacaaaagaatgaacaaattttctcaaatgtcacccattgtgctatggcttttagacttctaaggatgagctactatgatgtctcctcagatgaactagcagaatttgtggatgaagaacatttctttgcaacaaatgggaaatataaaagtcatgttgaaattcttgaactccacaaagcatcacaattggctattgatcatgagaaagatgacattttggataaaataaacaattggacaagagcttttatggagcaaaaactcttaaacaatggcttcatagataggatgtcaaagaaagaggtggaacttgctttgaggaagttttataccacatctcatctagcagaaaatagaagatatataaagtcatacgaagagaacaattttaaaatcttaaaagcagcttataggtcacccaacattaacaataaggacttgttagcattttcaatacacgactttgaattatgccaagctcaacacagagaagaacttcaacaactcaagaggtggtttgaagattatagattggaccaactcggacttgcagaaagatatatacatgctagttacttatttggtgttactgttatccccgagcctgaattatccgatgctagactcatgtacgcgaaatacgtcatgctcctgactattgtcgatgatcatttcgagagttttgcatctaaagatgaatgtttcaacatcattgaattagtagaaaggtgggatgactatgcaagtgtaggttataaatctgagaaggttaaagtttttttttctgttttctataaatcaatagaggagcttgcaacaattgctgaaattaaacaaggaagatccgtcaaaaatcaccttattaatttgtggcttgaattgatgaagttgatgttgatggagagagtagagtggtgttctggcaagacaataccaagcatagaagagtacttgtatgttacatctataacattttgtgcaaaattgattcctctctcaacacaatattttcttggaataaaaatatccaaagatctactagaaagtgatgaaatatgtggcctatggaattgtagcggtagagtgatgagaatccttaatgatttacaagattccaagagagaacaaaaggaggtctcaataaatttagtcacattactaatgaaaagtatgtctgaggaagaagctataatgaagataaaggaaatcttggaaatgaatagaagagagttattgaaaatggttttagttcaaaaaaagggaagccaattgcctcaattatgcaaagatatattttggaggacaagcaaatgggctcatttcacttattcacaaactgatggatatagaattgcagaggaaatgaagaatcacattgatgaagtcttttacaaaccactcaatcattaataatagcataaccccttggggcctctaaacgggtcttgaggggttttttggggccctcgactttttaacaaaatatattgataaaaataataatagtgggtataattaagttgttagagaaaacgtataaattagggataaactatggaacttatgaaatagattgaaatggtttatctgttaccccgtaggatccagaatttaaaaggagggattaaaatgtctgctcgtggactcaacaagatttcatgctcactcaacttacaaaccgaaaagctttgttatgaggataatgataatgatcttgatgaagaacttatgcctaaacacattgctttgataatggatggtaataggagatgggcaaaggataagggtttagaagtatatgaaggtcacaaacatattattccaaaattaaaagagatttgtgacatttcttctaaattgggaatacaaattatcactgcttttgcattctctactgaaaattggaaaagatccaaggaggaggttgatttcttgttgcaaatgttcgaagaaatctatgatgagttttcgaggtctggagtaagagtgtctattataggttgtaaatccgacctcccaatgacattacaaaaatgcatagcattaacagaagagactacaaagggcaacaaaggacttcaccttgtgattgcactaaactatggtggatattatgacatattgcaagcaacaaaaagcattgttaataaagcaatgaatggtttattagatgtagaagatatcaacaagaatttatttgatcaagaacttgaaagcaagtgtccaaatcctgatttacttataaggacaggaggtgaacaaagagttagtaactttttgttgtggcaattggcttacactgaattttacttcaccaacacattgtttcctgattttggagaggaagatcttaaagaggcaataatgaactttcaacaaaggcatagacgttttggtggacacacatattaataataataattaattcgaacagaaaaaataagtatttatataacggttaattgtaaggagggttttttatgcaaactgaacatgttattttattgaatgcacagggagttcctactggtactctggaaaagtatgccgcacatacagcagacacccgcttacatctcgctttctccagttggctgtttaatgccaaaggacaattattagttaccagaagagcactgagcaaaaaagcatggcctggcgtgtggactaactctgtttgtgggcatccacaactgggagaaagcaacgaagacgcagtgatcagaagatgtcgttatgagcttggcgtggaaattactcctcctgaatctatctatcctgactttagatacagagccaccgatcctagtggcattgtggaaaatgaagtgtgtcctgtatttgccgcaagaaccactagtgcattacagatcaatgatgatgaagtgatggattatcaatggtgtgatttagcagatgtattacatggtattgatgccactccttgggctttcagtccttggatggtgatgcaggcaacaaatagagaagccagaaaaagattatctgcatttacccagcttaaataaaaataagagttaccttaaatggtaactcttatttttttaatgtcctcgagcgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaattaaaaatgaagttttaaaacttcatttttaatttaaattaaaaatgaagttttatcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaatgagggtaaaaataaaaaaataaaaaaataaaaaaataaaaaaataaaaaaaataaaaaaataaaaaaataaaaaaataaaaaaataaaaaaataaaaaaataaaaaaataaaaaaatataaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctggttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaattttttcaattttaacaatactttttgtaatattatgtttaaatttaattgtatttttttcatataataaagccgttgaagtaaaccaatccattttccttatgatgttattattaaatttaagttttataataatatctttattatatttattgtttttaaaaaaactagtgaaatttccggctttattaaacttatttttaggaattttattttcattttcatctttacaggatttgattatatctttaaatatgttttatcaaatattatctttttctaaatttatatatatttttattatatttattattatatatattttatttttaagtttctttctaacagctattaaaaagaaacttaaaaataaaaacacgtactctaaaccaataaataaaactatttttattattgctgccttgattggaatagtttttagtaaaattaatttcaatattccacaatattatattataagctagctttgcattgtacttttcaatcgcttcacgaatgcggttatctccgaaagataaagtcttttcatcttccttgatgaagataagattttctccgtctccgccggcagaattgaagcggggtactacggtatcgtctgcgtcatcttccgttgtctgatagatgatagtcataggctcattttcttccgtttcggtaaaggggataggttcgccctttgagagcagggcggcgatggaaagcattaacttgcttttcccatcgcccggatctccctgcaatagcgtaactttgccaaacggaatatacggataccacagccactttacttctttcggctcgatttcacttgccttgatgatttcaagaggtacgctgaaattcatttcgttttcatttagtttcattttttcttgttctccttttctctgaaaatataaaaaccacagattgatactaaaaccttggttgtgttgcttttcggggcttaaatcaaggaaaaatccttgttttaagcctttcaaaaagaaacacaaggtctttgtactaacctgtggttatgtataaaattgtagattttagggtaacaaaaaacaccgtatttctacgatgtttttgcttaaatacttgtttttagttacagacaaacctgaagttatcatagtcctaaattatattatagatttccctttaaataccccatatatacatgcatttaaaaaactttctattatgctgcttaatataaaaattaaaccctgcatttacataatgctagggttcaatttttcatttatatttgcttttaaattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttctgtataaaacactaatatcaatttctgtggttatactaaaagtcgtttgttggttcaaataatgattaaatatctcttttctcttccaattgtctaaatcaattttattaaagttcatgttcatttcctccctttaaatttaacacaaaattacacacacttatactataatcctttttagttgtatttttcaataaaaatcattcaaaaatataacttttgataagaaatttcacaaattaaagtatcaaaaaattttgctagtcaatactttactcaatattatataatgtaaatcaaataagcaaaaatttaatctgaagatgcttagtgggaatttgtaccccttatcgatacaaattccccgtaggcgctagggacactttttcactcgttaaaaagttttgagaatattttatatttttgttcatgtaatcactccttcttaattacaaatttttagcatctaatttaacttcaattcctattatacaaaattttaagatactgcactatcaacacactcttaagtttgcttctaagtcttatttccataacttcttttacgtttccgggtacaattcgtaatcatgtcatagctgtttcctgtgtgaaattcttatccgctcacaattccacacaacatacgagccggaagcataaagtgtaaagcctggggtgcctaatgagtgagctaactcacattaattgcgttgcgctcactgcccgctttccagtcgggaaacctgtcgtgccagaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattac

SEQ ID NO: 13 - DNA, 18188 bp, Artificial
  Gene cluster
gaattcgagctcggtacccggggatcctctagagtcgacctgcaggcatgccctaggtcgactttttaacaaaatatattgataaaaataataatagtgggtataattaagttgttagagaaaacgtataaattagggataaactatggaacttatgaaatagattgaaatggtttatctgttaccccgtaggatccagaatttaaaaggagggattaaacatatgaatggtttcgaagatgcaagggatagaataagggaaagttttgggaaattagagttatctccttcttcctatgacacagcatgggtagctatggtcccttcaagacattcactaaatgagccatgttttccacaatgtttggattggattattgaaaatcaaagagaagatggatcttggggactaaaccctacccatccattgcttctaaaggactcactttcttccactcttgcatgtttgcttgcactaaccaaatggagagttggagatgagcaaatcaaaagaggtcttggcttcattgaaacgtatggttgggcagtagataacaaggatcaaatttcacctttaggatttgaagttatattttctagtatgatcaaatctgcagagaaattagatttaaatttgcctttgaatcttcatcttgtaaatttggtgaaatgcaaaagagattcaacaattaaaaggaatgttgaatatatgggtgaaggagttggtgaattatgtgattggaaggaaatgataaagttacatcaaagacaaaatggttcattatttgattcaccagccactactgcagctgccttgatttatcatcaacatgatcaaaaatgctatcaatatcttaattcaatcttccaacaacacaaaaattgggttcccactatgtatccaacaaaggtacattcattgctttgcttggttgatacacttcaaaatcttggagtacataggcattttaaatcagaaataaagaaagctctagatgaaatatacaggctatggcaacaaaagaatgaacaaattttctcaaatgtcacccattgtgctatggcttttagacttctaaggatgagctactatgatgtctcctcagatgaactagcagaatttgtggatgaagaacatttctttgcaacaaatgggaaatataaaagtcatgttgaaattcttgaactccacaaagcatcacaattggctattgatcatgagaaagatgacattttggataaaataaacaattggacaagagcttttatggagcaaaaactcttaaacaatggcttcatagataggatgtcaaagaaagaggtggaacttgctttgaggaagttttataccacatctcatctagcagaaaatagaagatatataaagtcatacgaagagaacaattttaaaatcttaaaagcagcttataggtcacccaacattaacaataaggacttgttagcattttcaatacacgactttgaattatgccaagctcaacacagagaagaacttcaacaactcaagaggtggtttgaagattatagattggaccaactcggacttgcagaaagatatatacatgctagttacttatttggtgttactgttatccccgagcctgaattatccgatgctagactcatgtacgcgaaatacgtcatgctcctgactattgtcgatgatcatttcgagagttttgcatctaaagatgaatgtttcaacatcattgaattagtagaaaggtgggatgactatgcaagtgtaggttataaatctgagaaggttaaagtttttttttctgttttctataaatcaatagaggagcttgcaacaattgctgaaattaaacaaggaagatccgtcaaaaatcaccttattaatttgtggcttgaattgatgaagttgatgttgatggagagagtagagtggtgttctggcaagacaataccaagcatagaagagtacttgtatgttacatctataacattttgtgcaaaattgattcctctctcaacacaatattttcttggaataaaaatatccaaagatctactagaaagtgatgaaatatgtggcctatggaattgtagcggtagagtgatgagaatccttaatgatttacaagattccaagagagaacaaaaggaggtctcaataaatttagtcacattactaatgaaaagtatgtctgaggaagaagctataatgaagataaaggaaatcttggaaatgaatagaagagagttattgaaaatggttttagttcaaaaaaagggaagccaattgcctcaattatgcaaagatatattttggaggacaagcaaatgggctcatttcacttattcacaaactgatggatatagaattgcagaggaaatgaagaatcacattgatgaagtcttttacaaaccactcaatcattaataatagcataaccccttggggcctctaaacgggtcttgaggggttttttgtcgactttttaacaaaatatattgataaaaataataatagtgggtataattaagttgttagagaaaacgtataaattagggataaactatggaacttatgaaatagattgaaatggtttatctgttaccccgtaggatccagaatttaaaaggagggattaaaatgtctgctcgtggactcaacaagatttcatgctcactcaacttacaaaccgaaaagctttgttatgaggataatgataatgatcttgatgaagaacttatgcctaaacacattgctttgataatggatggtaataggagatgggcaaaggataagggtttagaagtatatgaaggtcacaaacatattattccaaaattaaaagagatttgtgacatttcttctaaattgggaatacaaattatcactgcttttgcattctctactgaaaattggaaaagatccaaggaggaggttgatttcttgttgcaaatgttcgaagaaatctatgatgagttttcgaggtctggagtaagagtgtctattataggttgtaaatccgacctcccaatgacattacaaaaatgcatagcattaacagaagagactacaaagggcaacaaaggacttcaccttgtgattgcactaaactatggtggatattatgacatattgcaagcaacaaaaagcattgttaataaagcaatgaatggtttattagatgtagaagatatcaacaagaatttatttgatcaagaacttgaaagcaagtgtccaaatcctgatttacttataaggacaggaggtgaacaaagagttagtaactttttgttgtggcaattggcttacactgaattttacttcaccaacacattgtttcctgattttggagaggaagatcttaaagaggcaataatgaactttcaacaaaggcatagacgttttggtggacacacatattaataataataattaattcgaacagaaaaaataagtatttatataacggttaattgtaaggagggttttttatgcaaactgaacatgttattttattgaatgcacagggagttcctactggtactctggaaaagtatgccgcacatacagcagacacccgcttacatctcgctttctccagttggctgtttaatgccaaaggacaattattagttaccagaagagcactgagcaaaaaagcatggcctggcgtgtggactaactctgtttgtgggcatccacaactgggagaaagcaacgaagacgcagtgatcagaagatgtcgttatgagcttggcgtggaaattactcctcctgaatctatctatcctgactttagatacagagccaccgatcctagtggcattgtggaaaatgaagtgtgtcctgtatttgccgcaagaaccactagtgcattacagatcaatgatgatgaagtgatggattatcaatggtgtgatttagcagatgtattacatggtattgatgccactccttgggctttcagtccttggatggtgatgcaggcaacaaatagagaagccagaaaaagattatctgcatttacccagcttaaataattttaaaatataagtgatttagatattcataatatatttgggaggtaaattaatatgaaagaagttgtaatagctagtgcagtaagaacagcgattggatcttatggaaagtctcttaaggatgtaccagcagtagatttaggagctacagctataaaggaagcagttaaaaaagcaggaataaaaccagaggatgttaatgaagtcattttaggaaatgttcttcaagcaggtttaggacagaatccagcaagacaggcatcttttaaagcaggattaccagttgaaattccagctatgactattaataaggtttgtggttcaggacttagaacagttagcttagcagcacaaattataaaagcaggagatgctgacgtaataatagcaggtggtatggaaaatatgtctagagctccttacttagcgaataacgctagatggggatatagaatgggaaacgctaaatttgttgatgaaatgatcactgacggattgtgggatgcatttaatgattaccacatgggaataacagcagaaaacatagctgagagatggaacatttcaagagaagaacaagatgagtttgctcttgcatcacaaaaaaaagctgaagaagctataaaatcaggtcaatttaaagatgaaatagttcctgtagtaattaaaggcagaaagggagaaactgtagttgatacagatgagcaccctagatttggatcaactatagaaggacttgcaaaattaaaacctgccttcaaaaaagatggaacagttacagctggtaatgcatcaggattaaatgactgtgcagcagtacttgtaatcatgagtgcagaaaaagctaaagagcttggagtaaaaccacttgctaagatagtttcttatggttcagcaggagttgacccagcaataatgggatatggacctttctatgcaacaaaagcagctattgaaaaagcaggttggacagttgatgaattagatttaatagaatcaaatgaagcttttgcagctcaaagtttagcagtagcaaaagatttaaaatttgatatgaataaagtaaatgtaaatggaggagctattgcccttggtcatccaattggagcatcaggtgcaagaatactcgttactcttgtacacgcaatgcaaaaaagagatgcaaaaaaaggcttagcaactttatgtataggtggcggacaaggaacagcaatattgctagaaaagtgctagtagaaataagagttaccttaaatggtaactcttatttttttaatgtcacatagagaatttcactctttgcattttatctaacatcaaggggtttatttgtcacaaattatgtaaaaataaaacaaagatgtaagaaagtcctatgatataaattttgtaaacataataaattagctttcataagattggaagaatgataattactacttagaactgctaaaaattaggaaagaggtgtcgttaattaatgcagaaaagacaaagggagctgagtgcgttgacactacctacctctgctgagggggtatcagaaagccatagggcccgttctgtcggcatcggtcgtgcccatgccaaggccatcctgctgggagagcatgcggtagtatacggagcgccggcactcgctctgcctattcctcagctcacggtcacggccagcgttggctggtcttccgaggcctccgacagtgcgggtggcctgtcctacacgatgaccggtacgccttctagggcactggtgacgcaggcctccgacggcctgcataggctcaccgcggaattcatggcgaggatgggcgtgacgaacgcgcctcatctcgacgtgatcctggacggcgcgatccctcacggcaggggtctcggctccagcgcggccggctcacgtgcgatcgccttggccctcgccgacctcttcggccacgaactggccgagcatacggcgtacgaactggtgcagacggccgagaacatggcgcatggcagggccagcggcgtggacgcgatgacggtcggcgcgtccaggcctctgctgttccagcagggccgtaccgagagactggccatcggctgcgacagcctgttcatcgtagccgacagcggcgtacctggcagcaccaaggaagcggtagagatgctgagggagggattcacccgtagcgccggaacacaggagcggttcgttggcagggcgacggaactgaccgaggccgccaggcaggccctcgccgacggcaggcccgaggagctgggctctcagctgacgtactaccatgagctgctccatgaggcccgtctgagcaccgacggcatcgatgcgctggtagaggccgcgctgaaggcaggcagcctcggagccaagatcaccggcggtggtctgggcggctgcatgatcgcacaggccaggcccgaacaggccagggaggtaaccaggcagctccatgaggccggtgccgtacagacctgggtagtaccactgaaagggctcgacaaccatgcgcagtaataattttaaaatataagtgatttagatattcataatatatttgggaggtaaattaatatgcgtagtgaacatcctaccacgaccgtgctccagtctagggagcagggcagcgcggccggcgccaccgcggtagcgcatccaaacatcgcgctgatcaagtactggggcaagcgtgacgagaggctgatcctgccctgcaccaccagcctgtctatgacgctggacgtattccccacgaccaccgaggtcaggctcgaccccgccgccgagcatgacacggccgccctcaacggcgaggtggccacgggcgagacgctgcgtcgtatcagcgccttcctctccctggtgagggaggtggcgggcagcgaccagagggccgtggtggacacccgtaacaccgtgcccaccggggcgggcctggcgtcctccgccagcgggttcgccgccctcgccgtcgcggccgcggccgcctacgggctcgaactcgacgaccgtgggctgtccaggctggccagacgtggatccggctccgcctctcggtctatcttcggcggcttcgccgtatggcatgccggccccgacggcacggccacggaagcggacctcggctcctacgccgagccagtgcccgcggccgacctcgaccctgcgctggttatcgccgtggtaaacgccggccccaagcccgtatccagccgtgaggccatgcgtcgcaccgtagacacctcaccactgtacaggccatgggccgactccagtaaggacgacctggacgagatgcgttctgcgctgctgcgtggcgacctcgaggccgtgggcgagatcgcggagcgtaacgcgctcggcatgcatgccaccatgctggccgcccgtcccgcggtgaggtacctgtcaccagccacggtaaccgtgctcgacagcgtgctccagctccgtaaggacggtgttctggcctacgcgaccatggacgccggtcccaacgtgaaggtgctgtgcaggagggcggacgccgagagggtggccgacgttgtacgcgccgccgcgtccggcggtcaggtactcgtagccgggcctggagacggtgcccgtctgctgagcgagggcgcataataattttaaaatataagtgatttagatattcataatatatttgggaggtaaattaatatgacgacaggtcagcgtacgatcgtcaggcatgcgcctggcaagctgttcgtagcgggcgagtacgcggtagtggatcctggcaaccctgcgatcctggtagcggtagacaggcatatcagcgtaaccgtgtccgacgccgacgcggacaccggggccgccgacgtagtgatctcctccgacctcggtcctcaggcggtaggctggcgttggcatgacggcaggctcgtagtacgtgaccctgacgacgggcagcaggcgcgtagcgccctggcccatgtggtgtcggcgatcgagaccgtgggcaggctgctgggcgaacgtggacagaaggtacccgctctcaccctctccgttagcagccgtctgcatgaggacggcaggaagttcggcctgggctccagcggcgcggtgaccgtggcgaccgtagccgccgtagccgcgttctgcggactcgaactgtccaccgacgaaaggttcaggctggccatgctcgccaccgcggaactcgaccccaagggctccggcggggacctcgccgccagcacctggggcggctggatcgcctaccaggcgcccgacagggcctttgtgctcgacctggccaggcgtgtgggagtagacaggacactgaaggcgccctggccggggcattctgtgcgtagactgcctgcgcccaagggcctcaccctggaggtcggctggaccggagagcccgcctccaccgcgtccctggtgtccgatctgcatcgtcgtacctggaggggcagcgcctcccatcagaggttcgtagagaccacgaccgactgtgtacgttccgcggttaccgccctggagtccggcgacgacacgagcctgctgcatgagatccgcagggcccgtcaggagctggcccgtctggacgacgaggtaggcctcggcatcttcacacccaagctgacggcgctgtgcgacgccgccgaagccgttggcggcgcggccaagccctccggggcaggcggcggcgactgcggcatcgccctgctggacgccgaggcgtctagggacatcacacatgtaaggcaacggtgggagacagccggggtgctgcccctgcccctgactcctgccctggaagggatctaataattttaaaatataagtgatttagatattcataatatatttgggaggtaaattaatatgagcctcgattccagactgcccgctttccgtaacctgtcccctgccgcgagactggaccacatcggccagttgctcggcctgagccacgacgatgtcagcctgctggccaacgccggtgccctgccgatggacatcgccaacggcatgatcgaaaacgtcatcggcaccttcgagctgccctatgccgtggccagcaacttccagatcaatggccgtgatgtgctggtgccgctggtggtggaagagccctcgatcgtcgccgctgcttcttacatggccaagctggcccgtgccaacggcggcttcaccacctccagcagcgccccgctgatgcatgcccaggtacagatcgtcggcatacaggacccgctcaatgcacgtctgagcctgctgagaagaaaagacgaaatcattgaactggccaaccgtaaggaccagttgctcaacagcctcggcggcggctgcagagacatcgaagtgcacaccttcgccgataccccgcgtggcccgatgctggtggcgcacctgatcgtcgatgtaagagatgccatgggcgccaacaccgtcaataccatggccgaggccgttgcgccgctgatggaagccatcaccgggggccaggtacgtctgagaattctgtccaacctggccgacctgcgcctggccagggcccaggtgaggattactccgcagcaactggaaacggccgaattcagtggcgaggcagtgatcgaaggcatcctcgacgcctacgccttcgctgcggtcgacccttacagagcggccacccacaacaagggcatcatgaatggcatcgacccactgatcgtcgccactggcaacgactggcgtgcagtggaagccggcgcccatgcgtatgcctgcagaagtggtcactacggctcgctgaccacctgggaaaaggacaacaacggccatttggtcggcaccctggaaatgccgatgcccgtaggcctggtcggcggcgccaccaaaacccatccgctggcgcaactgtcactgagaatcctcggcgtgaaaacagcccaggcgctcgctgagattgccgtggccgtaggcctggcgcaaaacctcggggccatgagagccctggccaccgaaggcatccagcgtggccacatggccctgcatgcgagaaatattgccgtggtggcgggcgcccgaggcgatgaggtggactgggttgcccggcagttggtggaataccacgacgtgagagccgacagagccgtagcactgctgaaacaaaagagaggccaatgatagttttaaaatataagtgatttagatattcataatatatttgggaggtaaattaatatgtccatctccataggcattcacgacctgtctttcgccacaaccgagttcgtactgcctcatacggcgctcgccgagtacaacggcaccgagatcggcaagtaccatgtaggcatcggccagcagtctatgagcgtgcctgccgccgacgaggacatcgtgaccatggccgcgaccgcggcgaggcccatcatcgagcgtaacggcaagagcaggatccgtacggtagtgttcgccacggagtcttctatcgaccaggcgaaggcgggcggcgtatacgtgcactccctgctggggctggagtctgcctgcagggtagtagagctgaagcaggcctgctacggggccaccgccgcccttcagttcgccatcggcctggtgaggcgtgaccccgcccagcaggtactggtaatcgccagtgacgtatccaagtacgagctggacagccccggcgaggcgacccagggcgcggccgcggtggccatgctggtaggcgccgaccctgccctgctgcgtatcgaggagccttcgggcctgttcaccgccgacgtaatggacttctggcggcccaactacctcaccaccgctctggtagacggccaggagtccatcaacgcctacctgcaggccgtagagggcgcctggaaggactacgcggagcaggacggcaggtcactggaggagttcgcggcgttcgtataccaccagccgttcacgaagatggcctacaaggcgcatcgccatctgctgaacttcaacggctacgacaccgacaaggacgccatcgagggcgccctcggccagacgacggcgtacaacaacgtaatcggcaacagctacaccgcgtctgtgtacctgggcctggccgccctgctcgaccaggcggacgacctgacgggccgttccatcggcttcctgagctacggctctggcagcgtagccgagttcttctctggcaccgtagtagccgggtaccgtgagcgtctgcgtaccgaggcgaaccaggaggcgatcgccaggcgtaagagcgtagactacgccacctaccgtgagctgcacgagtacacgctcccgtccgacggcggcgaccatgccacccctgtgcagaccaccggccccttcaggctggccgggatcaacgaccataagcgtatctacgaggcgcgttaataatttaaaagcaaatataaatgaaaaattgaaccctagcattatgtaaatgcagggtttaatttttatattaagcagcataatagaaagttttttaaatgcatgtatatatggggtatttaaagggaaatctataatataatttaggactatacgcgtcgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaattaaaaatgaagttttaaaacttcatttttaatttaaattaaaaatgaagttttatcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaatgagggtaaaaataaaaaaataaaaaaataaaaaaataaaaaaataaaaaaaataaaaaaataaaaaaataaaaaaataaaaaaataaaaaaataaaaaaataaaaaaataaaaaaatataaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctggttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaattttttcaattttaacaatactttttgtaatattatgtttaaatttaattgtatttttttcatataataaagccgttgaagtaaaccaatccattttccttatgatgttattattaaatttaagttttataataatatctttattatatttattgtttttaaaaaaactagtgaaatttccggctttattaaacttatttttaggaattttattttcattttcatctttacaggatttgattatatctttaaatatgttttatcaaatattatctttttctaaatttatatatatttttattatatttattattatatatattttatttttaagtttctttctaacagctattaaaaagaaacttaaaaataaaaacacgtactctaaaccaataaataaaactatttttattattgctgccttgattggaatagtttttagtaaaattaatttcaatattccacaatattatattataagctagctttgcattgtacttttcaatcgcttcacgaatgcggttatctccgaaagataaagtcttttcatcttccttgatgaagataagattttctccgtctccgccggcagaattgaagcggggtactacggtatcgtctgcgtcatcttccgttgtctgatagatgatagtcataggctcattttcttccgtttcggtaaaggggataggttcgccctttgagagcagggcggcgatggaaagcattaacttgcttttcccatcgcccggatctccctgcaatagcgtaactttgccaaacggaatatacggataccacagccactttacttctttcggctcgatttcacttgccttgatgatttcaagaggtacgctgaaattcatttcgttttcatttagtttcattttttcttgttctccttttctctgaaaatataaaaaccacagattgatactaaaaccttggttgtgttgcttttcggggcttaaatcaaggaaaaatccttgttttaagcctttcaaaaagaaacacaaggtctttgtactaacctgtggttatgtataaaattgtagattttagggtaacaaaaaacaccgtatttctacgatgtttttgcttaaatacttgtttttagttacagacaaacctgaagttatcatagtcctaaattatattatagatttccctttaaataccccatatatacatgcatttaaaaaactttctattatgctgcttaatataaaaattaaaccctgcatttacataatgctagggttcaatttttcatttatatttgcttttaaattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttctgtataaaacactaatatcaatttctgtggttatactaaaagtcgtttgttggttcaaataatgattaaatatctcttttctcttccaattgtctaaatcaattttattaaagttcatgttcatttcctccctttaaatttaacacaaaattacacacacttatactataatcctttttagttgtatttttcaataaaaatcattcaaaaatataacttttgataagaaatttcacaaattaaagtatcaaaaaattttgctagtcaatactttactcaatattatataatgtaaatcaaataagcaaaaatttaatctgaagatgcttagtgggaatttgtaccccttatcgatacaaattccccgtaggcgctagggacactttttcactcgttaaaaagttttgagaatattttatatttttgttcatgtaatcactccttcttaattacaaatttttagcatctaatttaacttcaattcctattatacaaaattttaagatactgcactatcaacacactcttaagtttgcttctaagtcttatttccataacttcttttacgtttccgggtacaattcgtaatcatgtcatagctgtttcctgtgtgaaattcttatccgctcacaattccacacaacatacgagccggaagcataaagtgtaaagcctggggtgcctaatgagtgagctaactcacattaattgcgttgcgctcactgcccgctttccagtcgggaaacctgtcgtgccagaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattac

SEQ ID NO: 14 - DNA, 16239 bp, Artificial
  Gene cluster
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggagatcggtacttcgcgaatgcgtcgagatggcgcgccttggtaaaggatatatggtagtatttgcaggggatacatagggagatatagaagttctaccaataggttcagaatttagccatagttctgatctaaaagctgtactagacgtatttgatactgtaaaggtatttatatataacttaactcctgaatcctctgggtttatcaagcctccccaagcatttaaattacgttctagaattagaaagggggtatgacccataaaatattttcctttaaaagattcatatacacgatagggtaggtttactactttagttggtttatcatgatgacttacggtatcggaatttaaaatgttttgattttccataaatatgacctcctagtatttagtattattttatgtaaatatatatgtagaagtgtaccatttgtgcaagatttcaataaagggtatattttacctatttttttagtataaaaaatgcaaaaaatatgaacaaaagtagagttcctatgtattaaattgtaaaatatccactaaaaaaataaaattataataaaaaatacaaaaaaataattgacaatatataaataattatgcataattatatcatgataacaattagttaagcataattacatatatatgaacataatatgacatcttagaagcatatctttcgttagtaataatataatttcctttagaagaaaatgatttatttaaaataaatagtgtaatgttttttataatttcaaaaagttccccaatttagcatactaggcatgataaaaatagcttgaataagtgcccgggattatttattgatacatagagaatttcactctttgcattttatctaacatcaaggggtttatttgtcacaaattatgtaaaaataaaacaaagatgtaagaaagtcctatgatataaattttgtaaacataataaattagctttcataagattggaagaatgataattactacttagaactgctaaaaattaggaaagaggtgtcgttaattaatggaaaccagaaggtctgccaattatgaaccaaatagctgggattatgattatttgctgtcttctgacactgacgaatctattgaagtatacaaagacaaggccaaaaagctggaggctgaggtgagaagagagattaacaatgaaaaggcagagtttttgactctgcctgaactgatagataatgttcaaaggttaggattaggttacagattcgagagtgacataaggagagcccttgatagatttgtttcttcaggaggatttgatgctgttacaaaaactagccttcatgctactgctcttagcttcaggcttctcagacagcatggctttgaggtatctcaagaagctttcagcggattcaaggatcaaaatggcaatttcttgaaaaaccttaaggaggacatcaaggcaatactaagcctatatgaagcttcatttcttgccttagaaggagaaaatatcttggatgaggccaaggtgtttgcaatatcacatctaaaagagcttagcgaagaaaagattggaaaagacctggccgaacaggtgaatcatgcattggagcttccattgcatagaaggacacaaagactagaagctgtttggagcattgaagcatacagaaaaaaggaagatgcagatcaagtactgctagaacttgctatattggactacaacatgattcaatcagtataccaaagagatcttagagagacatcaaggtggtggaggagagtgggtcttgcaacaaagttgcattttgctagagacaggttaattgaaagcttttactgggcagttggagttgcatttgaacctcaatacagtgattgtagaaattccgtagcaaaaatgttttcttttgtaacaatcattgatgatatctatgatgtttatggtactctggatgagttggagctatttacagatgctgttgagagatgggatgttaatgccatcgatgatcttcctgattatatgaagctttgtttcctagctctttataacactatcaatgagatagcttatgataatctgaaggacaagggggaaaacattcttccatacctaacaaaagcatgggcagatttatgtaatgcattcctacaagaagcaaaatggttgtacaataagtccacaccaacatttgatgaatatttcggaaatgcatggaaatcatcctcagggcctcttcaactagtttttgcctactttgccgttgttcaaaacatcaagaaagaggaaattgataacttacaaaagtatcatgatatcatcagtaggccttcccatatctttagactttgtaacgacttggcttcagcatctgctgagatagcaagaggtgaaaccgcaaattctgtatcatgttacatgagaacaaaaggcatttctgaggaacttgctactgaatccgtaatgaatttgatcgacgaaacctggaaaaagatgaacaaagaaaagcttggtggctctctgtttgcaaaaccttttgttgaaacagctattaaccttgcaagacaatcccattgtacttatcataacggagatgcacatacttcaccagatgagcttactaggaaaagagtactgtcagtaatcacagagcctattctaccttttgagagataataattttaaaatataagtgatttagatattcataatatatttgggaggtaaattaatatgaccagcgcccaacgtaaggacgaccatgtaaggctcgccatcgagcagcataacgcccatagcggacgtaaccagttcgacgacgtgtctttcgtacatcatgccctggccggcatcgacaggccagacgtgtccctggccacgtccttcgccgggatctcctggcaggtgcctatctacatcaacgcgatgaccggcggcagcgagaagaccggcctcatcaacagggacctggccaccgccgcccgtgagaccggcgtacccatcgcgtccgggtccatgaacgcgtacatcaaggacccctcctgcgccgacacgttccgtgtgctgcgtgacgagaaccccaacgggttcgtaatcgcgaacatcaacgccaccacgacggttgacaacgcgcagcgtgcgatcgacctgatcgaggcgaacgccctgcagatccatatcaacacggcgcaggagacgcctatgcctgagggcgacaggtctttcgcgtcctgggtccctcagatcgagaagatcgcggcggccgtagacatccccgtgatcgtaaaggaggtaggcaacggcctgagcaggcagaccatcctgctgctcgccgacctcggcgtgcaggcggcggacgtaagcggccgtggcggcacggacttcgcccgtatcgagaacggccgtagggagctcggcgactacgcgttcctgcatggctgggggcagtccaccgccgcctgcctgctggacgcccaggacatctccctgcccgtactcgcctccggcggtgtgcgtcatcctctcgacgtggtacgtgccctcgcgctcggcgcccgtgccgtaggctcctccgccggcttcctgcgtaccctgatggacgacggcgtagacgcgctgatcacgaagctcacgacctggctggaccagctggcggcgctgcagaccatgctcggcgcgcgtacccctgccgacctcacccgttgcgacgtgctgctccatggcgagctgcgtgacttctgcgccgacaggggcatcgacacgcgtcgtctcgcccagcgttccagctccatcgaggccctccagacgacgggaagcacaagataataattttaaaatataagtgatttagatattcataatatatttgggaggtaaattaatatgaaagaagttgtaatagctagtgcagtaagaacagcgattggatcttatggaaagtctcttaaggatgtaccagcagtagatttaggagctacagctataaaggaagcagttaaaaaagcaggaataaaaccagaggatgttaatgaagtcattttaggaaatgttcttcaagcaggtttaggacagaatccagcaagacaggcatcttttaaagcaggattaccagttgaaattccagctatgactattaataaggtttgtggttcaggacttagaacagttagcttagcagcacaaattataaaagcaggagatgctgacgtaataatagcaggtggtatggaaaatatgtctagagctccttacttagcgaataacgctagatggggatatagaatgggaaacgctaaatttgttgatgaaatgatcactgacggattgtgggatgcatttaatgattaccacatgggaataacagcagaaaacatagctgagagatggaacatttcaagagaagaacaagatgagtttgctcttgcatcacaaaaaaaagctgaagaagctataaaatcaggtcaatttaaagatgaaatagttcctgtagtaattaaaggcagaaagggagaaactgtagttgatacagatgagcaccctagatttggatcaactatagaaggacttgcaaaattaaaacctgccttcaaaaaagatggaacagttacagctggtaatgcatcaggattaaatgactgtgcagcagtacttgtaatcatgagtgcagaaaaagctaaagagcttggagtaaaaccacttgctaagatagtttcttatggttcagcaggagttgacccagcaataatgggatatggacctttctatgcaacaaaagcagctattgaaaaagcaggttggacagttgatgaattagatttaatagaatcaaatgaagcttttgcagctcaaagtttagcagtagcaaaagatttaaaatttgatatgaataaagtaaatgtaaatggaggagctattgcccttggtcatccaattggagcatcaggtgcaagaatactcgttactcttgtacacgcaatgcaaaaaagagatgcaaaaaaaggcttagcaactttatgtataggtggcggacaaggaacagcaatattgctagaaaagtgctagtagtttaaaagcaaatataaatgaaaaattgaaccctagcattatgtaaatgcagggtttaatttttatattaagcagcataatagaaagttttttaaatgcatgtatatatggggtatttaaagggaaatctataatataatttaggactatacatagagaatttcactctttgcattttatctaacatcaaggggtttatttgtcacaaattatgtaaaaataaaacaaagatgtaagaaagtcctatgatataaattttgtaaacataataaattagctttcataagattggaagaatgataattactacttagaactgctaaaaattaggaaagaggtgtcgttaattaatgcagaaaagacaaagggagctgagtgcgttgacactacctacctctgctgagggggtatcagaaagccatagggcccgttctgtcggcatcggtcgtgcccatgccaaggccatcctgctgggagagcatgcggtagtatacggagcgccggcactcgctctgcctattcctcagctcacggtcacggccagcgttggctggtcttccgaggcctccgacagtgcgggtggcctgtcctacacgatgaccggtacgccttctagggcactggtgacgcaggcctccgacggcctgcataggctcaccgcggaattcatggcgaggatgggcgtgacgaacgcgcctcatctcgacgtgatcctggacggcgcgatccctcacggcaggggtctcggctccagcgcggccggctcacgtgcgatcgccttggccctcgccgacctcttcggccacgaactggccgagcatacggcgtacgaactggtgcagacggccgagaacatggcgcatggcagggccagcggcgtggacgcgatgacggtcggcgcgtccaggcctctgctgttccagcagggccgtaccgagagactggccatcggctgcgacagcctgttcatcgtagccgacagcggcgtacctggcagcaccaaggaagcggtagagatgctgagggagggattcacccgtagcgccggaacacaggagcggttcgttggcagggcgacggaactgaccgaggccgccaggcaggccctcgccgacggcaggcccgaggagctgggctctcagctgacgtactaccatgagctgctccatgaggcccgtctgagcaccgacggcatcgatgcgctggtagaggccgcgctgaaggcaggcagcctcggagccaagatcaccggcggtggtctgggcggctgcatgatcgcacaggccaggcccgaacaggccagggaggtaaccaggcagctccatgaggccggtgccgtacagacctgggtagtaccactgaaagggctcgacaaccatgcgcagtaataattttaaaatataagtgatttagatattcataatatatttgggaggtaaattaatatgcgtagtgaacatcctaccacgaccgtgctccagtctagggagcagggcagcgcggccggcgccaccgcggtagcgcatccaaacatcgcgctgatcaagtactggggcaagcgtgacgagaggctgatcctgccctgcaccaccagcctgtctatgacgctggacgtattccccacgaccaccgaggtcaggctcgaccccgccgccgagcatgacacggccgccctcaacggcgaggtggccacgggcgagacgctgcgtcgtatcagcgccttcctctccctggtgagggaggtggcgggcagcgaccagagggccgtggtggacacccgtaacaccgtgcccaccggggcgggcctggcgtcctccgccagcgggttcgccgccctcgccgtcgcggccgcggccgcctacgggctcgaactcgacgaccgtgggctgtccaggctggccagacgtggatccggctccgcctctcggtctatcttcggcggcttcgccgtatggcatgccggccccgacggcacggccacggaagcggacctcggctcctacgccgagccagtgcccgcggccgacctcgaccctgcgctggttatcgccgtggtaaacgccggccccaagcccgtatccagccgtgaggccatgcgtcgcaccgtagacacctcaccactgtacaggccatgggccgactccagtaaggacgacctggacgagatgcgttctgcgctgctgcgtggcgacctcgaggccgtgggcgagatcgcggagcgtaacgcgctcggcatgcatgccaccatgctggccgcccgtcccgcggtgaggtacctgtcaccagccacggtaaccgtgctcgacagcgtgctccagctccgtaaggacggtgttctggcctacgcgaccatggacgccggtcccaacgtgaaggtgctgtgcaggagggcggacgccgagagggtggccgacgttgtacgcgccgccgcgtccggcggtcaggtactcgtagccgggcctggagacggtgcccgtctgctgagcgagggcgcataataattttaaaatataagtgatttagatattcataatatatttgggaggtaaattaatatgacgacaggtcagcgtacgatcgtcaggcatgcgcctggcaagctgttcgtagcgggcgagtacgcggtagtggatcctggcaaccctgcgatcctggtagcggtagacaggcatatcagcgtaaccgtgtccgacgccgacgcggacaccggggccgccgacgtagtgatctcctccgacctcggtcctcaggcggtaggctggcgttggcatgacggcaggctcgtagtacgtgaccctgacgacgggcagcaggcgcgtagcgccctggcccatgtggtgtcggcgatcgagaccgtgggcaggctgctgggcgaacgtggacagaaggtacccgctctcaccctctccgttagcagccgtctgcatgaggacggcaggaagttcggcctgggctccagcggcgcggtgaccgtggcgaccgtagccgccgtagccgcgttctgcggactcgaactgtccaccgacgaaaggttcaggctggccatgctcgccaccgcggaactcgaccccaagggctccggcggggacctcgccgccagcacctggggcggctggatcgcctaccaggcgcccgacagggcctttgtgctcgacctggccaggcgtgtgggagtagacaggacactgaaggcgccctggccggggcattctgtgcgtagactgcctgcgcccaagggcctcaccctggaggtcggctggaccggagagcccgcctccaccgcgtccctggtgtccgatctgcatcgtcgtacctggaggggcagcgcctcccatcagaggttcgtagagaccacgaccgactgtgtacgttccgcggttaccgccctggagtccggcgacgacacgagcctgctgcatgagatccgcagggcccgtcaggagctggcccgtctggacgacgaggtaggcctcggcatcttcacacccaagctgacggcgctgtgcgacgccgccgaagccgttggcggcgcggccaagccctccggggcaggcggcggcgactgcggcatcgccctgctggacgccgaggcgtctagggacatcacacatgtaaggcaacggtgggagacagccggggtgctgcccctgcccctgactcctgccctggaagggatctaataattttaaaatataagtgatttagatattcataatatatttgggaggtaaattaatatgagcctcgattccagactgcccgctttccgtaacctgtcccctgccgcgagactggaccacatcggccagttgctcggcctgagccacgacgatgtcagcctgctggccaacgccggtgccctgccgatggacatcgccaacggcatgatcgaaaacgtcatcggcaccttcgagctgccctatgccgtggccagcaacttccagatcaatggccgtgatgtgctggtgccgctggtggtggaagagccctcgatcgtcgccgctgcttcttacatggccaagctggcccgtgccaacggcggcttcaccacctccagcagcgccccgctgatgcatgcccaggtacagatcgtcggcatacaggacccgctcaatgcacgtctgagcctgctgagaagaaaagacgaaatcattgaactggccaaccgtaaggaccagttgctcaacagcctcggcggcggctgcagagacatcgaagtgcacaccttcgccgataccccgcgtggcccgatgctggtggcgcacctgatcgtcgatgtaagagatgccatgggcgccaacaccgtcaataccatggccgaggccgttgcgccgctgatggaagccatcaccgggggccaggtacgtctgagaattctgtccaacctggccgacctgcgcctggccagggcccaggtgaggattactccgcagcaactggaaacggccgaattcagtggcgaggcagtgatcgaaggcatcctcgacgcctacgccttcgctgcggtcgacccttacagagcggccacccacaacaagggcatcatgaatggcatcgacccactgatcgtcgccactggcaacgactggcgtgcagtggaagccggcgcccatgcgtatgcctgcagaagtggtcactacggctcgctgaccacctgggaaaaggacaacaacggccatttggtcggcaccctggaaatgccgatgcccgtaggcctggtcggcggcgccaccaaaacccatccgctggcgcaactgtcactgagaatcctcggcgtgaaaacagcccaggcgctcgctgagattgccgtggccgtaggcctggcgcaaaacctcggggccatgagagccctggccaccgaaggcatccagcgtggccacatggccctgcatgcgagaaatattgccgtggtggcgggcgcccgaggcgatgaggtggactgggttgcccggcagttggtggaataccacgacgtgagagccgacagagccgtagcactgctgaaacaaaagagaggccaatgatagttttaaaatataagtgatttagatattcataatatatttgggaggtaaattaatatgtccatctccataggcattcacgacctgtctttcgccacaaccgagttcgtactgcctcatacggcgctcgccgagtacaacggcaccgagatcggcaagtaccatgtaggcatcggccagcagtctatgagcgtgcctgccgccgacgaggacatcgtgaccatggccgcgaccgcggcgaggcccatcatcgagcgtaacggcaagagcaggatccgtacggtagtgttcgccacggagtcttctatcgaccaggcgaaggcgggcggcgtatacgtgcactccctgctggggctggagtctgcctgcagggtagtagagctgaagcaggcctgctacggggccaccgccgcccttcagttcgccatcggcctggtgaggcgtgaccccgcccagcaggtactggtaatcgccagtgacgtatccaagtacgagctggacagccccggcgaggcgacccagggcgcggccgcggtggccatgctggtaggcgccgaccctgccctgctgcgtatcgaggagccttcgggcctgttcaccgccgacgtaatggacttctggcggcccaactacctcaccaccgctctggtagacggccaggagtccatcaacgcctacctgcaggccgtagagggcgcctggaaggactacgcggagcaggacggcaggtcactggaggagttcgcggcgttcgtataccaccagccgttcacgaagatggcctacaaggcgcatcgccatctgctgaacttcaacggctacgacaccgacaaggacgccatcgagggcgccctcggccagacgacggcgtacaacaacgtaatcggcaacagctacaccgcgtctgtgtacctgggcctggccgccctgctcgaccaggcggacgacctgacgggccgttccatcggcttcctgagctacggctctggcagcgtagccgagttcttctctggcaccgtagtagccgggtaccgtgagcgtctgcgtaccgaggcgaaccaggaggcgatcgccaggcgtaagagcgtagactacgccacctaccgtgagctgcacgagtacacgctcccgtccgacggcggcgaccatgccacccctgtgcagaccaccggccccttcaggctggccgggatcaacgaccataagcgtatctacgaggcgcgttaataatttaaaagcaaatataaatgaaaaattgaaccctagcattatgtaaatgcagggtttaatttttatattaagcagcataatagaaagttttttaaatgcatgtatatatggggtatttaaagggaaatctataatataatttaggactattccggataccgttcgtataatgtatgctatacgaagttatttcagattaaatttttgcttatttgatttacattatataatattgagtaaagtattgactagcaaaattttttgatactttaatttgtgaaatttcttatcaaaagttatatttttgaatgatttttattgaaaaatacaactaaaaaggattatagtataagtgtgtgtaattttgtgttaaatttaaagggaggaaatgaacatgaactttaataaaattgatttagacaattggaagagaaaagagatatttaatcattatttgaaccaacaaacgacttttagtataaccacagaaattgatattagtgttttatacagaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataataatttaaaagcaaatataaatgaaaaattgaaccctagcattatgtaaatgcagggtttaatttttatattaagcagcataatagaaagttttttaaatgcatgtatatatggggtatttaaagggaaatctataatataatttaggactatataacttcgtataatgtatgctatacgaacggtacctaggatatataataaattgaatatagtaaacaaaaagggacatatttataatatgttctttttagtttaatactcaatttttgcacataagaaattaacttaatataaaaaaatttgcgaagctttgcttcgcagtttaatattgtttaggtggttaaattatgaatctggaagtgttaaaaacagagtttaagtatttaagagataaaataattgaaaagcaatatgaacatcttgatcctatgcaaagaaaagcagttttaaatggtgaaaataactgtattgttattgcttgtcctggagcaggaaagacccagactattattaatagagtggactacttatgtagattcggtcctatatacaatacagattatgtacctaattgtctaaagaccgatgatttacagataatgaagaaatatttaaatgataattcttttaaagatgtgactgcagtaaataaaattgagcatttgttaaatagcaataaaataaatccacagaacatagttgttataacttttactagagcagctgctctcaatatgaaaaacagatacatatctataggaaataaagaaaagtcacctttttttggaacattccactccctattttataatatattgaaaaagcataataaagaaataaatattatagatccttataaggcacatgagatagttaaaaatacacttatgtattatctggactttataggagaagagagagtaaaggaagttctaaatgacatatctcttttaaaaaatagtgaaactaacatagatttatttaaaagtaaaattgacaaaagtgtatttttaaaatgttttaatgaatatgaaaattataaagctagaaataagcttatggattttgatgatttacaattaaaagttaaagatatgtttctaaatcagaaatctattctagatagttatcagaatttgttcaagtatattttagttgatgagtttcaggattcagataacctccaaatattcgaaatcggatgccgggaccgacgagtgcagaggcgtgcaagcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 15 - DNA, 17400 bp, Artificial
  Gene cluster
gaattcgagctcggtacccgggattatttattgatacatagagaatttcactctttgcattttatctaacatcaaggggtttatttgtcacaaattatgtaaaaataaaacaaagatgtaagaaagtcctatgatataaattttgtaaacataataaattagctttcataagattggaagaatgataattactacttagaactgctaaaaattaggaaagaggtgtcgttaattaatggaaaccagaaggtctgccaattatgaaccaaatagctgggattatgattatttgctgtcttctgacactgacgaatctattgaagtatacaaagacaaggccaaaaagctggaggctgaggtgagaagagagattaacaatgaaaaggcagagtttttgactctgcctgaactgatagataatgttcaaaggttaggattaggttacagattcgagagtgacataaggagagcccttgatagatttgtttcttcaggaggatttgatgctgttacaaaaactagccttcatgctactgctcttagcttcaggcttctcagacagcatggctttgaggtatctcaagaagctttcagcggattcaaggatcaaaatggcaatttcttgaaaaaccttaaggaggacatcaaggcaatactaagcctatatgaagcttcatttcttgccttagaaggagaaaatatcttggatgaggccaaggtgtttgcaatatcacatctaaaagagcttagcgaagaaaagattggaaaagacctggccgaacaggtgaatcatgcattggagcttccattgcatagaaggacacaaagactagaagctgtttggagcattgaagcatacagaaaaaaggaagatgcagatcaagtactgctagaacttgctatattggactacaacatgattcaatcagtataccaaagagatcttagagagacatcaaggtggtggaggagagtgggtcttgcaacaaagttgcattttgctagagacaggttaattgaaagcttttactgggcagttggagttgcatttgaacctcaatacagtgattgtagaaattccgtagcaaaaatgttttcttttgtaacaatcattgatgatatctatgatgtttatggtactctggatgagttggagctatttacagatgctgttgagagatgggatgttaatgccatcgatgatcttcctgattatatgaagctttgtttcctagctctttataacactatcaatgagatagcttatgataatctgaaggacaagggggaaaacattcttccatacctaacaaaagcatgggcagatttatgtaatgcattcctacaagaagcaaaatggttgtacaataagtccacaccaacatttgatgaatatttcggaaatgcatggaaatcatcctcagggcctcttcaactagtttttgcctactttgccgttgttcaaaacatcaagaaagaggaaattgataacttacaaaagtatcatgatatcatcagtaggccttcccatatctttagactttgtaacgacttggcttcagcatctgctgagatagcaagaggtgaaaccgcaaattctgtatcatgttacatgagaacaaaaggcatttctgaggaacttgctactgaatccgtaatgaatttgatcgacgaaacctggaaaaagatgaacaaagaaaagcttggtggctctctgtttgcaaaaccttttgttgaaacagctattaaccttgcaagacaatcccattgtacttatcataacggagatgcacatacttcaccagatgagcttactaggaaaagagtactgtcagtaatcacagagcctattctaccttttgagagataataattttaaaatataagtgatttagatattcataatatatttgggaggtaaattaatatgaccagcgcccaacgtaaggacgaccatgtaaggctcgccatcgagcagcataacgcccatagcggacgtaaccagttcgacgacgtgtctttcgtacatcatgccctggccggcatcgacaggccagacgtgtccctggccacgtccttcgccgggatctcctggcaggtgcctatctacatcaacgcgatgaccggcggcagcgagaagaccggcctcatcaacagggacctggccaccgccgcccgtgagaccggcgtacccatcgcgtccgggtccatgaacgcgtacatcaaggacccctcctgcgccgacacgttccgtgtgctgcgtgacgagaaccccaacgggttcgtaatcgcgaacatcaacgccaccacgacggttgacaacgcgcagcgtgcgatcgacctgatcgaggcgaacgccctgcagatccatatcaacacggcgcaggagacgcctatgcctgagggcgacaggtctttcgcgtcctgggtccctcagatcgagaagatcgcggcggccgtagacatccccgtgatcgtaaaggaggtaggcaacggcctgagcaggcagaccatcctgctgctcgccgacctcggcgtgcaggcggcggacgtaagcggccgtggcggcacggacttcgcccgtatcgagaacggccgtagggagctcggcgactacgcgttcctgcatggctgggggcagtccaccgccgcctgcctgctggacgcccaggacatctccctgcccgtactcgcctccggcggtgtgcgtcatcctctcgacgtggtacgtgccctcgcgctcggcgcccgtgccgtaggctcctccgccggcttcctgcgtaccctgatggacgacggcgtagacgcgctgatcacgaagctcacgacctggctggaccagctggcggcgctgcagaccatgctcggcgcgcgtacccctgccgacctcacccgttgcgacgtgctgctccatggcgagctgcgtgacttctgcgccgacaggggcatcgacacgcgtcgtctcgcccagcgttccagctccatcgaggccctccagacgacgggaagcacaagataataattttaaaatataagtgatttagatattcataatatatttgggaggtaaattaatatgaaagaagttgtaatagctagtgcagtaagaacagcgattggatcttatggaaagtctcttaaggatgtaccagcagtagatttaggagctacagctataaaggaagcagttaaaaaagcaggaataaaaccagaggatgttaatgaagtcattttaggaaatgttcttcaagcaggtttaggacagaatccagcaagacaggcatcttttaaagcaggattaccagttgaaattccagctatgactattaataaggtttgtggttcaggacttagaacagttagcttagcagcacaaattataaaagcaggagatgctgacgtaataatagcaggtggtatggaaaatatgtctagagctccttacttagcgaataacgctagatggggatatagaatgggaaacgctaaatttgttgatgaaatgatcactgacggattgtgggatgcatttaatgattaccacatgggaataacagcagaaaacatagctgagagatggaacatttcaagagaagaacaagatgagtttgctcttgcatcacaaaaaaaagctgaagaagctataaaatcaggtcaatttaaagatgaaatagttcctgtagtaattaaaggcagaaagggagaaactgtagttgatacagatgagcaccctagatttggatcaactatagaaggacttgcaaaattaaaacctgccttcaaaaaagatggaacagttacagctggtaatgcatcaggattaaatgactgtgcagcagtacttgtaatcatgagtgcagaaaaagctaaagagcttggagtaaaaccacttgctaagatagtttcttatggttcagcaggagttgacccagcaataatgggatatggacctttctatgcaacaaaagcagctattgaaaaagcaggttggacagttgatgaattagatttaatagaatcaaatgaagcttttgcagctcaaagtttagcagtagcaaaagatttaaaatttgatatgaataaagtaaatgtaaatggaggagctattgcccttggtcatccaattggagcatcaggtgcaagaatactcgttactcttgtacacgcaatgcaaaaaagagatgcaaaaaaaggcttagcaactttatgtataggtggcggacaaggaacagcaatattgctagaaaagtgctagtagtttaaaagcaaatataaatgaaaaattgaaccctagcattatgtaaatgcagggtttaatttttatattaagcagcataatagaaagttttttaaatgcatgtatatatggggtatttaaagggaaatctataatataatttaggactatacatagagaatttcactctttgcattttatctaacatcaaggggtttatttgtcacaaattatgtaaaaataaaacaaagatgtaagaaagtcctatgatataaattttgtaaacataataaattagctttcataagattggaagaatgataattactacttagaactgctaaaaattaggaaagaggtgtcgttaattaatgcagaaaagacaaagggagctgagtgcgttgacactacctacctctgctgagggggtatcagaaagccatagggcccgttctgtcggcatcggtcgtgcccatgccaaggccatcctgctgggagagcatgcggtagtatacggagcgccggcactcgctctgcctattcctcagctcacggtcacggccagcgttggctggtcttccgaggcctccgacagtgcgggtggcctgtcctacacgatgaccggtacgccttctagggcactggtgacgcaggcctccgacggcctgcataggctcaccgcggaattcatggcgaggatgggcgtgacgaacgcgcctcatctcgacgtgatcctggacggcgcgatccctcacggcaggggtctcggctccagcgcggccggctcacgtgcgatcgccttggccctcgccgacctcttcggccacgaactggccgagcatacggcgtacgaactggtgcagacggccgagaacatggcgcatggcagggccagcggcgtggacgcgatgacggtcggcgcgtccaggcctctgctgttccagcagggccgtaccgagagactggccatcggctgcgacagcctgttcatcgtagccgacagcggcgtacctggcagcaccaaggaagcggtagagatgctgagggagggattcacccgtagcgccggaacacaggagcggttcgttggcagggcgacggaactgaccgaggccgccaggcaggccctcgccgacggcaggcccgaggagctgggctctcagctgacgtactaccatgagctgctccatgaggcccgtctgagcaccgacggcatcgatgcgctggtagaggccgcgctgaaggcaggcagcctcggagccaagatcaccggcggtggtctgggcggctgcatgatcgcacaggccaggcccgaacaggccagggaggtaaccaggcagctccatgaggccggtgccgtacagacctgggtagtaccactgaaagggctcgacaaccatgcgcagtaataattttaaaatataagtgatttagatattcataatatatttgggaggtaaattaatatgcgtagtgaacatcctaccacgaccgtgctccagtctagggagcagggcagcgcggccggcgccaccgcggtagcgcatccaaacatcgcgctgatcaagtactggggcaagcgtgacgagaggctgatcctgccctgcaccaccagcctgtctatgacgctggacgtattccccacgaccaccgaggtcaggctcgaccccgccgccgagcatgacacggccgccctcaacggcgaggtggccacgggcgagacgctgcgtcgtatcagcgccttcctctccctggtgagggaggtggcgggcagcgaccagagggccgtggtggacacccgtaacaccgtgcccaccggggcgggcctggcgtcctccgccagcgggttcgccgccctcgccgtcgcggccgcggccgcctacgggctcgaactcgacgaccgtgggctgtccaggctggccagacgtggatccggctccgcctctcggtctatcttcggcggcttcgccgtatggcatgccggccccgacggcacggccacggaagcggacctcggctcctacgccgagccagtgcccgcggccgacctcgaccctgcgctggttatcgccgtggtaaacgccggccccaagcccgtatccagccgtgaggccatgcgtcgcaccgtagacacctcaccactgtacaggccatgggccgactccagtaaggacgacctggacgagatgcgttctgcgctgctgcgtggcgacctcgaggccgtgggcgagatcgcggagcgtaacgcgctcggcatgcatgccaccatgctggccgcccgtcccgcggtgaggtacctgtcaccagccacggtaaccgtgctcgacagcgtgctccagctccgtaaggacggtgttctggcctacgcgaccatggacgccggtcccaacgtgaaggtgctgtgcaggagggcggacgccgagagggtggccgacgttgtacgcgccgccgcgtccggcggtcaggtactcgtagccgggcctggagacggtgcccgtctgctgagcgagggcgcataataattttaaaatataagtgatttagatattcataatatatttgggaggtaaattaatatgacgacaggtcagcgtacgatcgtcaggcatgcgcctggcaagctgttcgtagcgggcgagtacgcggtagtggatcctggcaaccctgcgatcctggtagcggtagacaggcatatcagcgtaaccgtgtccgacgccgacgcggacaccggggccgccgacgtagtgatctcctccgacctcggtcctcaggcggtaggctggcgttggcatgacggcaggctcgtagtacgtgaccctgacgacgggcagcaggcgcgtagcgccctggcccatgtggtgtcggcgatcgagaccgtgggcaggctgctgggcgaacgtggacagaaggtacccgctctcaccctctccgttagcagccgtctgcatgaggacggcaggaagttcggcctgggctccagcggcgcggtgaccgtggcgaccgtagccgccgtagccgcgttctgcggactcgaactgtccaccgacgaaaggttcaggctggccatgctcgccaccgcggaactcgaccccaagggctccggcggggacctcgccgccagcacctggggcggctggatcgcctaccaggcgcccgacagggcctttgtgctcgacctggccaggcgtgtgggagtagacaggacactgaaggcgccctggccggggcattctgtgcgtagactgcctgcgcccaagggcctcaccctggaggtcggctggaccggagagcccgcctccaccgcgtccctggtgtccgatctgcatcgtcgtacctggaggggcagcgcctcccatcagaggttcgtagagaccacgaccgactgtgtacgttccgcggttaccgccctggagtccggcgacgacacgagcctgctgcatgagatccgcagggcccgtcaggagctggcccgtctggacgacgaggtaggcctcggcatcttcacacccaagctgacggcgctgtgcgacgccgccgaagccgttggcggcgcggccaagccctccggggcaggcggcggcgactgcggcatcgccctgctggacgccgaggcgtctagggacatcacacatgtaaggcaacggtgggagacagccggggtgctgcccctgcccctgactcctgccctggaagggatctaataattttaaaatataagtgatttagatattcataatatatttgggaggtaaattaatatgagcctcgattccagactgcccgctttccgtaacctgtcccctgccgcgagactggaccacatcggccagttgctcggcctgagccacgacgatgtcagcctgctggccaacgccggtgccctgccgatggacatcgccaacggcatgatcgaaaacgtcatcggcaccttcgagctgccctatgccgtggccagcaacttccagatcaatggccgtgatgtgctggtgccgctggtggtggaagagccctcgatcgtcgccgctgcttcttacatggccaagctggcccgtgccaacggcggcttcaccacctccagcagcgccccgctgatgcatgcccaggtacagatcgtcggcatacaggacccgctcaatgcacgtctgagcctgctgagaagaaaagacgaaatcattgaactggccaaccgtaaggaccagttgctcaacagcctcggcggcggctgcagagacatcgaagtgcacaccttcgccgataccccgcgtggcccgatgctggtggcgcacctgatcgtcgatgtaagagatgccatgggcgccaacaccgtcaataccatggccgaggccgttgcgccgctgatggaagccatcaccgggggccaggtacgtctgagaattctgtccaacctggccgacctgcgcctggccagggcccaggtgaggattactccgcagcaactggaaacggccgaattcagtggcgaggcagtgatcgaaggcatcctcgacgcctacgccttcgctgcggtcgacccttacagagcggccacccacaacaagggcatcatgaatggcatcgacccactgatcgtcgccactggcaacgactggcgtgcagtggaagccggcgcccatgcgtatgcctgcagaagtggtcactacggctcgctgaccacctgggaaaaggacaacaacggccatttggtcggcaccctggaaatgccgatgcccgtaggcctggtcggcggcgccaccaaaacccatccgctggcgcaactgtcactgagaatcctcggcgtgaaaacagcccaggcgctcgctgagattgccgtggccgtaggcctggcgcaaaacctcggggccatgagagccctggccaccgaaggcatccagcgtggccacatggccctgcatgcgagaaatattgccgtggtggcgggcgcccgaggcgatgaggtggactgggttgcccggcagttggtggaataccacgacgtgagagccgacagagccgtagcactgctgaaacaaaagagaggccaatgatagttttaaaatataagtgatttagatattcataatatatttgggaggtaaattaatatgtccatctccataggcattcacgacctgtctttcgccacaaccgagttcgtactgcctcatacggcgctcgccgagtacaacggcaccgagatcggcaagtaccatgtaggcatcggccagcagtctatgagcgtgcctgccgccgacgaggacatcgtgaccatggccgcgaccgcggcgaggcccatcatcgagcgtaacggcaagagcaggatccgtacggtagtgttcgccacggagtcttctatcgaccaggcgaaggcgggcggcgtatacgtgcactccctgctggggctggagtctgcctgcagggtagtagagctgaagcaggcctgctacggggccaccgccgcccttcagttcgccatcggcctggtgaggcgtgaccccgcccagcaggtactggtaatcgccagtgacgtatccaagtacgagctggacagccccggcgaggcgacccagggcgcggccgcggtggccatgctggtaggcgccgaccctgccctgctgcgtatcgaggagccttcgggcctgttcaccgccgacgtaatggacttctggcggcccaactacctcaccaccgctctggtagacggccaggagtccatcaacgcctacctgcaggccgtagagggcgcctggaaggactacgcggagcaggacggcaggtcactggaggagttcgcggcgttcgtataccaccagccgttcacgaagatggcctacaaggcgcatcgccatctgctgaacttcaacggctacgacaccgacaaggacgccatcgagggcgccctcggccagacgacggcgtacaacaacgtaatcggcaacagctacaccgcgtctgtgtacctgggcctggccgccctgctcgaccaggcggacgacctgacgggccgttccatcggcttcctgagctacggctctggcagcgtagccgagttcttctctggcaccgtagtagccgggtaccgtgagcgtctgcgtaccgaggcgaaccaggaggcgatcgccaggcgtaagagcgtagactacgccacctaccgtgagctgcacgagtacacgctcccgtccgacggcggcgaccatgccacccctgtgcagaccaccggccccttcaggctggccgggatcaacgaccataagcgtatctacgaggcgcgttaataatttaaaagcaaatataaatgaaaaattgaaccctagcattatgtaaatgcagggtttaatttttatattaagcagcataatagaaagttttttaaatgcatgtatatatggggtatttaaagggaaatctataatataatttaggactattccggagcatgcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaattaaaaatgaagttttaaaacttcatttttaatttaaattaaaaatgaagttttatcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaatgagggtaaaaataaaaaaataaaaaaataaaaaaataaaaaaataaaaaaaataaaaaaataaaaaaataaaaaaataaaaaaataaaaaaataaaaaaataaaaaaataaaaaaatataaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctggttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaattttttcaattttaacaatactttttgtaatattatgtttaaatttaattgtatttttttcatataataaagccgttgaagtaaaccaatccattttccttatgatgttattattaaatttaagttttataataatatctttattatatttattgtttttaaaaaaactagtgaaatttccggctttattaaacttatttttaggaattttattttcattttcatctttacaggatttgattatatctttaaatatgttttatcaaatattatctttttctaaatttatatatatttttattatatttattattatatatattttatttttaagtttctttctaacagctattaaaaagaaacttaaaaataaaaacacgtactctaaaccaataaataaaactatttttattattgctgccttgattggaatagtttttagtaaaattaatttcaatattccacaatattatattataagctagctttgcattgtacttttcaatcgcttcacgaatgcggttatctccgaaagataaagtcttttcatcttccttgatgaagataagattttctccgtctccgccggcagaattgaagcggggtactacggtatcgtctgcgtcatcttccgttgtctgatagatgatagtcataggctcattttcttccgtttcggtaaaggggataggttcgccctttgagagcagggcggcgatggaaagcattaacttgcttttcccatcgcccggatctccctgcaatagcgtaactttgccaaacggaatatacggataccacagccactttacttctttcggctcgatttcacttgccttgatgatttcaagaggtacgctgaaattcatttcgttttcatttagtttcattttttcttgttctccttttctctgaaaatataaaaaccacagattgatactaaaaccttggttgtgttgcttttcggggcttaaatcaaggaaaaatccttgttttaagcctttcaaaaagaaacacaaggtctttgtactaacctgtggttatgtataaaattgtagattttagggtaacaaaaaacaccgtatttctacgatgtttttgcttaaatacttgtttttagttacagacaaacctgaagttatcatagtcctaaattatattatagatttccctttaaataccccatatatacatgcatttaaaaaactttctattatgctgcttaatataaaaattaaaccctgcatttacataatgctagggttcaatttttcatttatatttgcttttaaattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttctgtataaaacactaatatcaatttctgtggttatactaaaagtcgtttgttggttcaaataatgattaaatatctcttttctcttccaattgtctaaatcaattttattaaagttcatgttcatttcctccctttaaatttaacacaaaattacacacacttatactataatcctttttagttgtatttttcaataaaaatcattcaaaaatataacttttgataagaaatttcacaaattaaagtatcaaaaaattttgctagtcaatactttactcaatattatataatgtaaatcaaataagcaaaaatttaatctgaagatgcttagtgggaatttgtaccccttatcgatacaaattccccgtaggcgctagggacactttttcactcgttaaaaagttttgagaatattttatatttttgttcatgtaatcactccttcttaattacaaatttttagcatctaatttaacttcaattcctattatacaaaattttaagatactgcactatcaacacactcttaagtttgcttctaagtcttatttccataacttcttttacgtttccgggtacaattcgtaatcatgtcatagctgtttcctgtgtgaaattcttatccgctcacaattccacacaacatacgagccggaagcataaagtgtaaagcctggggtgcctaatgagtgagctaactcacattaattgcgttgcgctcactgcccgctttccagtcgggaaacctgtcgtgccagaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattac

(19) United States
(12) Patent Application Publication
(10) Pub. No.: US 2018/0334657 A1
(43) Pub. Date: Nov. 22, 2018

(54) RECOMBINANT CELL, METHOD FOR PRODUCING RECOMBINANT CELL, AND METHOD FOR PRODUCING ORGANIC COMPOUND

(71) Applicant: SEKISUI CHEMICAL CO., LTD., Osaka-shi, Osaka (JP)

(72) Inventors: Masahiro FURUTANI, Arakawa-ku, Tokyo (JP); Norihide NISHIYAMA, Tsurugashima-shi, Saitama (JP); Kazufumi KAWABATA, Tsukuba-shi, Ibaraki (JP)

(73) Assignee: SEKISUI CHEMICAL CO., LTD., Osaka-shi, Osaka (JP)

(21) Appl. No.: 15/777,754

(22) PCT Filed: Nov. 30, 2015
(86) PCT No.: PCT/JP2015/083545

§ 371 Date: May 21, 2018

(51) Int. Cl. C12N 9/04; C12P 5/00; C12N 9/88; C12N 9/02; C12N 15/90

(52) U.S. Cl. CPC C12N 9/0006; C12P 5/002; C12P 5/007; C12N 9/88; C12Y 402/03027; C12N 15/902; C12N 9/0008; C12Y 101/01034; C12Y 101/01088; C12Y 402/03051; C12Y 102/0101; C12Y 101/01001

(57) ABSTRACT

A recombinant cell having a function of synthesizing acetyl-CoA from methyltetrahydrofolate, carbon monoxide, and CoA, including: a gene that expresses an exogenous NAD(P)H consumption pathway, the gene being expressed in the recombinant cell, wherein expression in at least one of endogenous NAD(P)H consumption pathways of the recombinant cell is down-regulated, and the endogenous NAD(P)H consumption pathway is different from the exogenous NAD(P)H consumption pathway, and the recombinant cell produces an organic compound having 4 or more carbon atoms from at least one selected from the group consisting of carbon monoxide and carbon dioxide via the exogenous NAD(P)H consumption pathway.

### TECHNICAL FIELD

The present invention relates to a recombinant cell capable of producing an organic compound having 4 or more carbon atoms from specific C1 compounds such as carbon monoxide, a method for manufacturing the same, and a method for producing the organic compound having 4 or more carbon atoms using the recombinant cell.

### BACKGROUND ART

Syngas (synthesis gas) is a mixed gas mainly containing carbon monoxide, carbon dioxide, and hydrogen, which is efficiently obtained from waste, natural gas and coal by action of a metal catalyst under high temperature and high pressure. In the field of C1 chemistry by metal catalysts starting from syngas, a process for mass production of liquid chemicals such as methanol, formic acid and formaldehyde at a low cost has been developed.

Carbon monoxide and carbon dioxide are contained in syngas derived from waste, industrial exhaust gas, or syngas derived from natural gas, and are available almost permanently. However, at present, there are very few examples of producing chemicals by microorganisms from these carbon sources. Only production of ethanol, 2,3-butanediol or the like, is currently under development. In particular, there are few reports about utilization of syngas assimilating substance by a recombinant. Patent Document 1 discloses a production technique of isopropanol by a recombinant of Escherichia coli. In this technique, a plurality of CO metabolic enzyme genes are introduced into E. coli to impart syngas assimilating ability, and isopropanol is produced from syngas.

Well-known examples of a Clostridium bacterium and a Moorella bacterium include syngas assimilating bacterium capable of assimilating carbon monoxide or carbon dioxide included in the syngas. The syngas assimilating bacterium is known to have carbon monoxide dehydrogenase (CO dehydrogenase, CODH) (for example, EC 1.2.99.2/1.2.7.4) having action of generating carbon dioxide and proton from carbon monoxide and water, and its reverse reaction, that is, action of generating carbon monoxide and water from carbon dioxide and proton. The carbon monoxide dehydrogenase is one of enzymes acting in an acetyl-CoA pathway (see FIG. 1).

Acetyl-CoA synthesized in the acetyl-CoA pathway is metabolized into acetic acid, acetaldehyde, ethanol, and the like. In particular, most of excessive acetyl-CoA in the proliferation stationary phase is converted into ethanol. At this time, reduced nicotinamide adenine dinucleotide (NADH) is consumed. In the process in which acetaldehyde is produced from acetyl-CoA and ethanol is produced from acetaldehyde, NADH is consumed in any cases, and oxidized nicotinamide adenine dinucleotide (NAD) is generated. Furthermore, similarly, nicotinamide adenine dinucleotide phosphate in which a phosphate group is attached at position 2′ of nucleotide of adenosine of nicotinamide adenine dinucleotide includes a reduced type (NADPH) and an oxidized type (NADP), and is known to work as a coenzyme of dehydrogenase. The balance of NAD(P)H/NAD(P) in a cell affects the growth. Non-Patent Document 1 mentions that Clostridium acetbutylicum cannot grow in glycerol metabolism because of excessive NADPH, and that introduction of a gene of 1,3-propanediol NADP-dependent dehydrogenase (YqhD) synthase that consumes NADPH permits growth and, at the same time, 1,3-propanediol is synthesized.

Some attempts for producing an organic compound having C4 or more from specific C1 compounds such as carbon monoxide by recombinant have been reported. Patent Documents 2 and 3 disclose an example in which an isoprene synthesis has been attempted using a Clostridium bacterium into which a gene of a mevalonate pathway that is an NAD(P)H consumption pathway (also referred to as an MVA pathway) in addition to an isoprene synthesis gene. However, in any cases, production amount of isoprene is very small.

Patent Document 4 discloses a recombinant cell capable of producing isoprene from a specific C1 compound such as carbon monoxide. It discloses an example in which isoprene is produced from syngas using a recombinant of a Clostridium bacterium.

The mevalonate pathway is a pathway to synthesize isopentenyl diphosphate (IPP) and dimethylallyl diphosphate (DMAPP), and is a starting point of synthesis of industrially applicable compounds such as terpene and steroid.

Terpene is a group of hydrocarbons having isoprene as a constituent unit. Terpene includes isoprene (the number of carbon atoms: 5), monoterpene (the number of carbon atoms: 10), sesquiterpene (the number of carbon atoms: 15), diterpene (the number of carbon atoms: 20), and triterpene (the number of carbon atoms: 30). Isoprene is a monomer raw material for synthetic polyisoprene, and is an important material, in particular, in a tire industry. As the cyclic monoterpene, β-Pinene, α-Pinene, limonene, α-Phellandrene, and the like, are considered to be a monomer raw material of adhesives or transparent resin (Non-Patent Document 3). Much attention has been paid on farnesene as one type of sesquiterpene as a raw material of tire (Patent Document 5). As triterpene, glycyrrhizic acid known as a licorice extract has been known.

### PRIOR ART DOCUMENTS

### Patent Documents

Patent Document 1: WO2009/094485
Patent Document 2: WO2013/181647
Patent Document 3: WO2013/180584
Patent Document 4: WO2014/065271
Patent Document 5: WO2013/047348

### Non-Patent Documents

Non-Patent Document 1: Tang X, Tan Y, Zhu H, Zhao K, Shen W., Appl Environ Microbiol. 2009 March; 75(6): 1628-34. doi: 10.1128/AEM.02376-08. Epub 2009 Jan. 9.
Non-Patent Document 2: Kiriukhin M, Tyurin M., Bioprocess Biosyst Eng. 2014 February; 37(2): 245-60. doi: 10.1007/s00449-013-0991-6. Epub 2013 Jun. 18
Non-Patent Document 3: Schilmiller, A. L., et al., Proc Natl Acad Sci USA., 2009.

### DISCLOSURE OF INVENTION

### Technical Problem

As mentioned above, technology development for producing an organic compound such as isoprene from syngas using a recombinant has been advanced, but techniques for improving productivity are demanded. Then, an object of the present invention is to provide a series of techniques for obtaining organic compounds of C4 or more from C1 carbon source such as syngas by using a recombinant at high efficiency.

### Solution to Problem

One aspect of the present invention for solving the aforementioned problem is a recombinant cell having a function of synthesizing acetyl-CoA from methyltetrahydrofolate, carbon monoxide, and CoA. The recombinant cell includes a gene that expresses an exogenous NAD(P)H consumption pathway, and the gene is expressed in the recombinant cell. Expression in at least one of endogenous NAD(P)H consumption pathways of the recombinant cell is down-regulated, and the endogenous NAD(P)H consumption pathway is different from the exogenous NAD(P)H consumption pathway. The recombinant cell produces an organic compound having 4 or more carbon atoms from at least one selected from the group consisting of carbon monoxide and carbon dioxide via the exogenous NAD(P)H consumption pathway.

This aspect relates to a recombinant cell capable of producing an organic compound having 4 or more carbon atoms. The recombinant cell of this aspect has a “function of synthesizing acetyl-CoA from methyltetrahydrofolate, carbon monoxide, and CoA”. Furthermore, the recombinant cell of this aspect includes a “gene that expresses an exogenous NAD(P)H consumption pathway,” and the gene is expressed in the recombinant cell. In other words, in the recombinant cell of this aspect, an NAD(P)H consumption pathway is newly added or enhanced.

Furthermore, in the recombinant cell of this aspect, expression of at least one of the endogenous NAD(P)H consumption pathways is down-regulated, and the endogenous NAD(P)H consumption pathway is different from that of the exogenous NAD(P)H consumption pathway.

The recombinant cell of this aspect produces an organic compound having 4 or more carbon atoms from at least one selected from the group consisting of carbon monoxide and carbon dioxide via the exogenous NAD(P)H consumption pathway.

In the recombinant cell of this aspect, since the expression of an endogenous NAD(P)H consumption pathway that is not involved in production of the organic compound, among the NAD(P)H consumption pathways, is down-regulated, the NAD(P)H is preferentially supplied to the exogenous NAD(P)H consumption pathway involved in production of the organic compound. Therefore, production of the organic compound is efficiently carried out. For example, by culturing the recombinant cell of this aspect, the organic compound having 4 or more carbon atoms can be mass-produced.

Examples of the cell having the “function of synthesizing acetyl-CoA from methyltetrahydrofolate, carbon monoxide, and CoA” in this aspect include anaerobic microorganisms having an acetyl-CoA pathway (Wood-Ljungdahl pathway) and a methanol pathway shown in FIG. 1.

The NAD(P)H represents NADH or NADPH. Similarly, the NAD(P) represents NAD or NADP.

The NAD(P)H consumption pathway means a pathway involving conversion of NAD(P)H to NAD(P), and in which NAD(P)H is consumed. The exogenous NAD(P)H consumption pathway means an NAD(P)H consumption pathway that is introduced into the host cell from the outside. The endogenous NAD(P)H consumption pathway represents an NAD(P)H consumption pathway which the host cell originally has.

The down-regulation of gene expression means regulation for reducing an expression amount of a gene, and regulation for impairing a function of a gene. Examples of the down-regulation of gene expression include a gene knockout and a gene knockdown. Furthermore, the gene knockout includes deletion of a gene itself.

Preferably, the recombinant cell is a Clostridium bacterium or a Moorella bacterium.

Preferably, the recombinant cell is a Clostridium ljungdahlii, Clostridium autoethanogenum, Clostridium carboxidivorans, Clostridium ragsdalei, Clostridium kluyveri, or Moorella thermoacetica.

Preferably, the exogenous NAD(P)H consumption pathway is a mevalonate pathway.

With such a configuration, a synthesis ability of isopentenyl diphosphate (IPP) and dimethylallyl diphosphate (DMAPP) is improved.

Preferably, the mevalonate pathway is a mevalonate pathway of yeast, prokaryote, or actinomycete.

Preferably, the mevalonate pathway is a mevalonate pathway of actinomycete.

Preferably, HMG-CoA reductase of the mevalonate pathway includes NADH-dependent HMG-CoA reductase.

Preferably, the HMG-CoA reductase is mvaA (P13702) derived from Pseudomonas mevalonii, hmgA-1Mtc_0274 (H8I942) derived from Methanocella conradii, mvaA LLKF_1694 (D2BKK7) derived from Lactococcus lactis subsp. lactis (strain KF147), or mvaA SSA_0337 (A3CKT9) derived from Streptococcus sanguinis (strain SK36).

Preferably, in the endogenous NAD(P)H consumption pathway, expression of at least one selected from the group consisting of ethanol dehydrogenase, acetaldehyde dehydrogenase, lactate dehydrogenase, and 2,3-butanediol dehydrogenase is down-regulated.

Examples of the endogenous NAD(P)H consumption pathway in Clostridium bacterium and the like include pathways for carrying out conversion from acetaldehyde to ethanol, conversion from acetyl-CoA to acetaldehyde, conversion from pyruvate to lactate, and conversion from pyruvate to 2,3-butanediol, respectively. Then, in this aspect, expression of enzymes that work in these endogenous NAD(P)H consumption pathways are down-regulated.

Preferably, in the endogenous NAD(P)H consumption pathway, expression of at least ethanol dehydrogenase is down-regulated.

Preferably, in the endogenous NAD(P)H consumption pathway, expression of acetaldehyde dehydrogenase is further down-regulated.

Preferably, in the endogenous NAD(P)H consumption pathway, expression of lactate dehydrogenase or 2,3-butanediol dehydrogenase is further down-regulated.

Preferably, at least one of the down-regulations is a lack of expression.

Preferably, the expression of ethanol dehydrogenase and/or acetaldehyde dehydrogenase lacks.

Preferably, a gene that expresses the mevalonate pathway is incorporated in a genome instead of a gene encoding ethanol dehydrogenase and/or acetaldehyde dehydrogenase, by homologous recombination.

Preferably, the expression of phosphotransacetylase and/or acetate kinase is down-regulated.

Phosphotransacetylase and acetate kinase are enzymes acting on pathway for converting acetyl-CoA to acetic acid. According to this aspect, wasteful consumption of acetyl-CoA is suppressed.

Preferably, expression of at least phosphotransacetylase is down-regulated.

Preferably, at least one of the down-regulations is carried out by gene deletion or modification of a gene expression-regulating region.

Preferably, the gene that expresses an exogenous NAD(P)H consumption pathway is a gene cluster that includes a gene that expresses a mevalonate pathway and a gene encoding an enzyme for producing isoprene from isopentenyl diphosphate, and the organic compound is isoprene.

With such a configuration, a recombinant cell capable of mass-producing isoprene is provided.

Preferably, an enzyme for producing isoprene from the isopentenyl diphosphate is isoprene synthase.

Preferably, the gene that expresses an exogenous NAD(P)H consumption pathway is a gene cluster that includes a gene that expresses a mevalonate pathway and a gene encoding an enzyme for producing cyclic terpene from isopentenyl diphosphate, and the organic compound is cyclic terpene.

With such a configuration, a recombinant cell capable of mass-producing cyclic terpene is provided.

Preferably, the enzyme for producing cyclic terpene from isopentenyl diphosphate is geranyl diphosphate synthase and/or neryl diphosphate synthase, and cyclic monoterpene synthase, and the organic compound is cyclic monoterpene.

Preferably, the cyclic monoterpene synthase is β-phellandrene synthase, and the cyclic monoterpene is β-phellandrene, 4-carene, or limonene.

According to this aspect, by the action of β-phellandrene synthase expressed in the cell, β-phellandrene, 4-carene, or limonene is synthesized from geranyl diphosphate (GPP) and/or neryl diphosphate (NPP). As a result, by culturing the recombinant cell of this aspect, β-phellandrene, 4-carene, or limonene can be mass-produced.

Preferably, the gene cluster is incorporated in a genome of the recombinant cell.

Preferably, a part or whole of an adhE1 gene and an adhE2 gene of a host cell that is a basis of the recombinant cell is deleted, and the gene cluster is incorporated in a genome, instead of the adhE1 gene and the adhE2 gene, by homologous recombination.

The adhE1 gene and the adhE2 gene are included in a Clostridium bacterium and the like, and are considered to include both an acetaldehyde dehydrogenase gene and an ethanol dehydrogenase gene.

Another aspect of the present invention relates to a method for manufacturing a recombinant cell. The method includes a first step of providing a host cell having a function of synthesizing acetyl-CoA from methyltetrahydrofolate, carbon monoxide, and CoA; and a second step of introducing a gene that expresses an exogenous NAD(P)H consumption pathway into the host cell. The gene that has been introduced in the second step is expressed in the host cell. Expression in at least one of endogenous NAD(P)H consumption pathways of the host cell is down-regulated, and the endogenous NAD(P)H consumption pathway is different from the exogenous NAD(P)H consumption pathway. The recombinant cell produces the organic compound having 4 or more carbon atoms from at least one selected from the group consisting of carbon monoxide and carbon dioxide, via the exogenous NAD(P)H consumption pathway.

This aspect relates to a method for manufacturing a recombinant cell capable of producing an organic compound having 4 or more carbon atoms. The method of this aspect includes a first step of providing a host cell having a function of synthesizing acetyl-CoA from methyltetrahydrofolate, carbon monoxide, and CoA; and a second step of introducing gene that expresses an exogenous NAD(P)H consumption pathway into the host cell. Then, the gene that has been introduced in the second step is expressed in the host cell. In other words, in the recombinant cell manufactured by the method of this aspect, an NAD(P)H consumption pathway is newly added or enhanced in the host cell.

Furthermore, in the recombinant cell manufactured by the method of this aspect, expression in at least one of the endogenous NAD(P)H consumption pathways of the host cell is down-regulated, and the endogenous NAD(P)H consumption pathway is different from the exogenous NAD(P)H consumption pathway.

The recombinant cell manufactured by the method of this aspect produces the organic compound having 4 or more carbon atoms from at least one selected from the group consisting of carbon monoxide and carbon dioxide, via the exogenous NAD(P)H consumption pathway.

In the recombinant cell manufactured by the method of this aspect, since the expression of an endogenous NAD(P)H consumption pathway that is not involved in production of the organic compound, among the NAD(P)H consumption pathways, is down-regulated, NAD(P)H is preferentially supplied to the exogenous NAD(P)H pathway that is involved in the organic compound. Therefore, production of the organic compound is carried out efficiently. For example, by culturing the recombinant cell of this aspect, the organic compound having 4 or more carbon atoms can be mass-produced.

Preferably, the host cell is a Clostridium bacterium or a Moorella bacterium.

Preferably, the host cell is Clostridium ljungdahlii, Clostridium autoethanogenum, Clostridium carboxidivorans, Clostridium ragsdalei, Clostridium kluyveri, or Moorella thermoacetica.

Preferably, the exogenous NAD(P)H consumption pathway is a mevalonate pathway.

Preferably, the mevalonate pathway is a mevalonate pathway of yeast, prokaryote, or actinomycete.

Preferably, the mevalonate pathway is a mevalonate pathway of actinomycete.

Preferably, HMG-CoA reductase of the mevalonate pathway includes NADH-dependent HMG-CoA reductase.

Preferably, the HMG-CoA reductase is mvaA (P13702) derived from Pseudomonas mevalonii, hmgA-1Mtc_0274 (H8I942) derived from Methanocella conradii, mvaA LLKF_1694 (D2BKK7) derived from Lactococcus lactis subsp. lactis (strain KF147), or mvaA SSA_0337 (A3CKT9) derived from Streptococcus sanguinis (strain SK36).

Preferably, in the endogenous NAD(P)H consumption pathway, expression of at least one selected from the group consisting of ethanol dehydrogenase, acetaldehyde dehydrogenase, lactate dehydrogenase, and 2,3-butanediol dehydrogenase is down-regulated.

Preferably, in the endogenous NAD(P)H consumption pathway, expression of at least ethanol dehydrogenase is down-regulated.

Preferably, in the endogenous NAD(P)H consumption pathway, expression of acetaldehyde dehydrogenase is further down-regulated.

Preferably, in the endogenous NAD(P)H consumption pathway, expression of lactate dehydrogenase or 2,3-butanediol dehydrogenase is further down-regulated.

Preferably, at least one of the down-regulations is a lack of expression.

Preferably, expression of ethanol dehydrogenase and/or acetaldehyde dehydrogenase lacks.

Preferably, a gene that expresses the mevalonate pathway is incorporated in a genome of the recombinant cell instead of a gene encoding ethanol dehydrogenase and/or acetaldehyde dehydrogenase, by homologous recombination.

Preferably, the expression of phosphotransacetylase and/or acetate kinase is further down-regulated.

Preferably, expression of at least phosphotransacetylase is down-regulated.

Preferably, at least one of the down-regulations is carried out by gene deletion or modification of a gene expression-regulating region.

Preferably, the gene that has been introduced in the second step is a gene cluster that includes a gene that expresses a mevalonate pathway and a gene encoding an enzyme for producing isoprene from isopentenyl diphosphate, and the organic compound is isoprene.

Preferably, an enzyme for producing isoprene from the isopentenyl diphosphate is isoprene synthase.

Preferably, the gene that has been introduced in the second step is a gene cluster that includes a gene that expresses a mevalonate pathway and a gene encoding an enzyme for producing cyclic terpene from isopentenyl diphosphate, and the organic compound is cyclic terpene.

Preferably, the enzyme for producing cyclic terpene from the isopentenyl diphosphate is geranyl diphosphate synthase and/or neryl diphosphate synthase, and cyclic monoterpene synthase, and the organic compound is cyclic monoterpene.

Preferably, the cyclic monoterpene synthase is β-phellandrene synthase, and the cyclic monoterpene is β-phellandrene, 4-carene, or limonene.

Preferably, the gene cluster is incorporated in a genome of a host cell.

Preferably, a part or whole of an adhE1 gene and an adhE2 gene of a host cell is deleted, and the gene cluster is incorporated in a genome, instead of the adhE1 gene and the adhE2 gene, by homologous recombination.

Another aspect of the present invention is a method for producing an organic compound. The method includes bringing at least one C1 compound selected from the group consisting of carbon monoxide and carbon dioxide into contact with the above-mentioned recombinant cell or a recombinant cell manufactured by the above-mentioned method to produce an organic compound having 4 or more carbon atoms from the C1 compound in the recombinant cell.

This aspect relates to a method for producing an organic compound having 4 or more carbon atoms. In this aspect, at least one C1 compound selected from the group consisting of carbon monoxide and carbon dioxide is brought into contact with the above-mentioned recombinant cell or the recombinant cell manufactured by the above-mentioned to produce the organic compound from the C1 compound. According to the method of this aspect, for example, it is possible to produce the organic compound from syngas including carbon monoxide or carbon dioxide.

Preferably, the recombinant cell is cultured using at least one C1 compound selected from the group consisting of carbon monoxide and carbon dioxide as a carbon source to produce an organic compound having 4 or more carbon atoms in the recombinant cell.

Preferably, the recombinant cell is provided with a gas mainly containing carbon monoxide, gas mainly containing carbon monoxide and hydrogen, gas mainly containing carbon dioxide and hydrogen, or gas mainly containing carbon monoxide, carbon dioxide and hydrogen.

The wording “provide a recombinant cell with a gas” means bringing the gas into contact with the recombinant cell. Examples thereof include providing the recombinant cell with gas as carbon source and the like when the recombinant cell is cultured.

Preferably, formic acid or methanol is further provided to the recombinant cell.

The wording “provide a recombinant cell with a gas and methanol” means bringing a gas and methanol into contact with a recombinant cell. Examples thereof include providing a recombinant cell with a gas and methanol as carbon source and the like in culturing the recombinant cell.

Preferably, the recombinant cell is a Clostridium bacterium or a Moorella bacterium.

Preferably, the organic compound released to outside the recombinant cell is collected.

Preferably, the organic compound is collected from a gas phase of a culture system of the recombinant cell.

### Effect of Invention

The present invention permits production of an organic compound having 4 or more carbon atoms by a recombinant via an exogenous NAD(P)H consumption pathway with high efficiency. In particular, with a configuration in which a mevalonate pathway is introduced as the exogenous NAD(P)H consumption pathway, isoprene or cyclic monoterpene can be produced by a recombinant with high efficiency.

### BRIEF DESCRIPTION OF DRAWINGS

FIG. 1 is an explanatory diagram showing an acetyl-CoA pathway and a methanol pathway.

FIG. 2 is an explanatory diagram showing a part of central metabolic pathway in a Clostridium bacterium or a Moorella bacterium in which a mevalonate pathway has been introduced.

### BEST MODE FOR CARRYING OUT THE INVENTION

Hereinafter, the exemplary embodiment of the present invention will be described. Note here that in the present invention, all the terms “gene” can be replaced with terms “nucleic acid” or “DNA”.

A recombinant cell of the present invention has a function of synthesizing acetyl-CoA from methyltetrahydrofolate, carbon monoxide, and CoA. The recombinant cell includes a gene that expresses an exogenous NAD(P)H consumption pathway. The gene is expressed in the recombinant cell. Expression in at least one of endogenous NAD(P)H consumption pathways of the recombinant cell is down-regulated, and the endogenous NAD(P)H consumption pathway is different from the exogenous NAD(P)H consumption pathway. The recombinant cell produces an organic compound having 4 or more carbon atoms from at least one selected from the group consisting of carbon monoxide and carbon dioxide via the exogenous NAD(P)H consumption pathway.

For example, the recombinant cell of the present invention is prepared by introducing a gene that expresses an exogenous NAD(P)H consumption pathway into a host cell having a function of synthesizing acetyl-CoA from methyltetrahydrofolate, carbon monoxide, and CoA, and the gene is expressed in the host cell. In at least one endogenous NAD(P)H consumption pathway of the host cell, the expression is down-regulated. The endogenous NAD(P)H consumption pathway is different from the exogenous NAD(P)H consumption pathway. The recombinant cell produces an organic compound having 4 or more carbon atoms from at least one selected from the group consisting of carbon monoxide and carbon dioxide via the exogenous NAD(P)H consumption pathway.

A method for manufacturing a recombinant cell of the present invention includes a first step of providing a host cell having a function of synthesizing acetyl-CoA from methyltetrahydrofolate, carbon monoxide, and CoA, and a second step of introducing a gene that expresses an exogenous NAD(P)H consumption pathway into the host cell. The gene that has been introduced in the second step is expressed in the host cell. In at least one endogenous NAD(P)H consumption pathway of the host cell, the expression is down-regulated, and the endogenous NAD(P)H consumption pathway is different from the exogenous NAD(P)H consumption pathway. The recombinant cell is capable of producing an organic compound having 4 or more carbon atoms from at least one selected from the group consisting of carbon monoxide and carbon dioxide via the exogenous NAD(P)H consumption pathway.

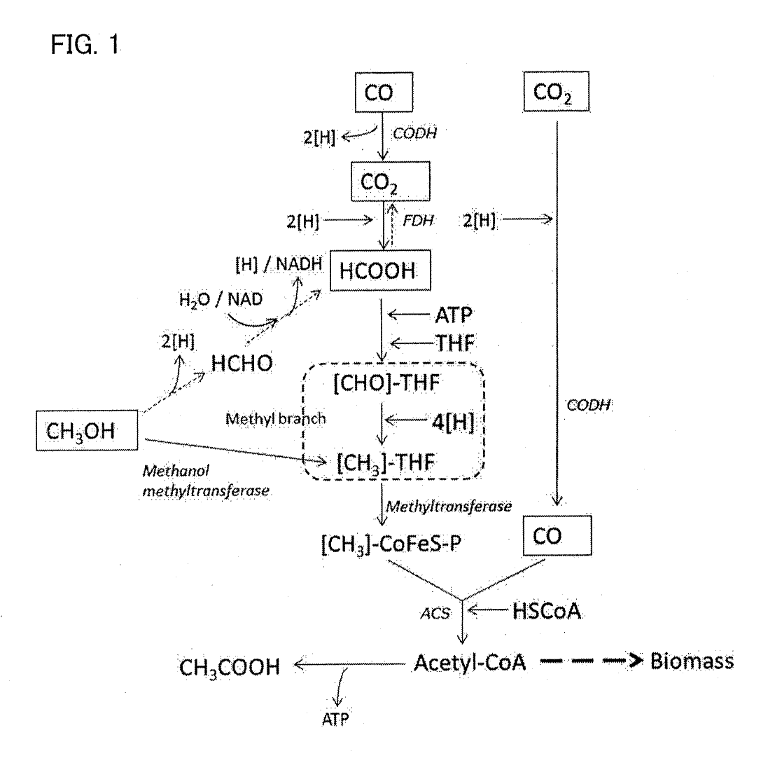

The recombinant cell of the present invention has a function of synthesizing acetyl-CoA from methyltetrahydrofolate, carbon monoxide, and CoA. For example, a host cell that is a basis of the recombinant cell of the present invention has a function of synthesizing acetyl-CoA from methyltetrahydrofolate, carbon monoxide, and CoA. A pathway for synthesizing acetyl-CoA from methyltetrahydrofolate ([CH₃]-THF), carbon monoxide (CO), and CoA is included in, for example, an acetyl-CoA pathway (Wood-Ljungdahl pathway) and a methanol pathway (Methanol pathway) shown in FIG. 1.

As shown in FIG. 1, in the acetyl-CoA pathway, carbon dioxide (CO₂) is reduced into carbon monoxide (CO) and a methyl cation source in two pathways, separately. Then, a thiol group of CoA (described as HSCoA in FIG. 1) is acetylated using these two carbon sources as a substrate, and one molecule of acetyl-CoA is synthesized. Enzymes such as an acetyl-CoA synthase (ACS), a methyltransferase, a carbon monoxide dehydrogenase (CODH), a formate dehydrogenase (FDH) act in the acetyl-CoA pathway. Note here that the pathway from formyltetrahydrofolate ([CHO]-THF) to [CH₃]-THF is referred to as methyl branch.

On the other hand, the methanol pathway includes a pathway for converting methanol into formaldehyde (HCHO), and further into formic acid (HCOOH), and a pathway for inducing from methanol to [CH₃]-THF.

That is to say, a pathway in which acetyl-CoA is synthesized from methyltetrahydrofolate ([CH₃]-THF), carbon monoxide (CO), and CoA is in common in the acetyl-CoA pathway and the methanol pathway.

Examples of the host cell include anaerobic microorganism such as a Clostridium bacterium and a Moorella bacterium. Specific examples include a Clostridium bacterium or a Moorella bacterium, such as Clostridium ljungdahlii, Clostridium autoethanogenum, Clostridium carboxidivorans, Clostridium ragsdalei (Kopke M. et al., Appl. Environ. Microbiol. 2011, 77(15), 5467-5475), Clostridium kluyveri, Moorella thermoacetica (that is the same as Clostridium thermoaceticum) (Pierce E G. et al., Environ. Microbiol. 2008, 10, 2550-2573). In particular, Clostridium bacteria are preferable as the host cell because their host-vector systems and culture methods have been established.

Other examples include Acetobacterium bacteria such as Acetobacterium woodii (Dilling S. et al., Appl. Environ. Microbiol. 2007, 73(11), 3630-3636). Furthermore, bacteria such as Carboxydocella sporoducens sp. November (Slepova T V et al., Inter. J. Sys. Evol. Microbiol. 2006, 56, 797-800), Rhodopseudomonas gelatinosa (Uffen R L, J. Bacteriol. 1983, 155(3), 956-965), Eubacterium limosum (Roh H. et al., J. Bacteriol. 2011, 193(1), 307-308), Butyribacterium methylotrophicum (Lynd, L H. et al., J. Bacteriol. 1983, 153(3), 1415-1423) are also examples of the host cell.

The recombinant cell of the present invention may have carbon monoxide dehydrogenase (CODH). In detail, a cell that grows mainly by carbon monoxide metabolism, that is, by a function of generating carbon dioxide and proton from carbon monoxide and water by the action of carbon monoxide dehydrogenase is preferred. The anaerobic microorganism having the acetyl-CoA pathway and methanol pathway mentioned above includes carbon monoxide dehydrogenase (CODH).

All of proliferation and CODH activity of the bacteria mentioned above are oxygen sensitive. However, oxygen insensitive CODH is also known. For example, oxygen insensitive CODH exists in other bacterial species represented by Oligotropha carboxidovorans (Schubel, U. et al., J. Bacteriol., 1995, 2197-2203), and Bradyrhizobium japonicum (Lorite M J. et al., Appl. Environ. Microbiol., 2000, 66(5), 1871-1876) (King G M et al., Appl. Environ. Microbiol. 2003, 69 (12), 7257-7265). Also in Ralsotonia bacteria which are aerobic hydrogen oxidizing bacteria, oxygen insensitive CODH exists (NCBI Gene ID: 4249199, 8019399).

As described above, bacteria having CODH widely exist. The host cell for use in the present invention can be appropriately selected from such bacteria. For example, using a selective medium containing CO, CO/H₂ (gas mainly containing CO and H₂), or CO/CO₂/H₂ (gas mainly containing CO, CO₂ and H₂) as the only carbon source and energy source, a bacterium having CODH that is usable as the host cell can be isolated in anaerobic, microaerobic or aerobic conditions.

As mentioned above, the NAD(P)H consumption pathway means a pathway involving conversion from NAD(P)H to NAD(P), and in which NAD(P)H is consumed. Examples of the NAD(P)H consumption pathways include a pathway in which NAD(P)H works as a coenzyme of dehydrogenase.

The recombinant cell of the present invention includes a gene that expresses an exogenous NAD(P)H consumption pathway, and the gene is expressed. For example, a gene that expresses an exogenous NAD(P)H consumption pathway is introduced into a host cell, and the gene is expressed. In a preferable embodiment, the host cell is a Clostridium bacterium or a Moorella bacterium, the exogenous NAD(P)H consumption pathway is a mevalonate pathway.

Synthesis pathways of isopentenyl diphosphate (IPP) are generally classified into a mevalonate pathway (MVA pathway) and a non-mevalonate pathway (MEP pathway). Among them, the mevalonate pathway is inherent in eukaryotes, and uses acetyl-CoA as a starting substance. Enzymes acting in the mevalonate pathway include acetyl-CoA acetyl transferase, HMG-CoA synthase, HMG-CoA reductase, mevalonate kinase, 5-phosphomevalonate kinase, diphosphomevalonate decarboxylase, and isopentenyl diphosphate isomerase, in the order from the upstream.

When IPP is synthesized from acetyl-CoA via the mevalonate pathway, NAD(P)H is consumed.

A gene that expresses the mevalonate pathway is a gene encoding these enzymes and synthesizing IPP from acetyl-CoA by the expression of the gene.

Note here that the mevalonate pathway is inherent in all eukaryotes, but is found also in prokaryotes. Examples of prokaryotes having a mevalonate pathway include, with respect to actinomycetes, Streptomyces sp. Strain CL190 (Takagi M. et al., J. Bacteriol. 2000, 182 (15), 4153-7), and Streptomyces griseolosporeus MF730-N6 (Hamano Y. et al., Biosci. Biotechnol. Biochem. 2001, 65(7), 1627-35).

Examples thereof include, with respect to bacteria, Lactobacillus helvecticus (Smeds A et al., DNA seq. 2001, 12(3), 187-190), Corynebacterium amycolatum, Mycobacterium marinum, Bacillus coagulans, Enterococcus faecalis, Streptococuss agalactiae, Myxococcus xanthus and the like (Lombard J. et al., Mol. Biol. Evol. 2010, 28 (1), 87-99).

Examples thereof include, with respect to archaea, genus Aeropyrum, genus Sulfolobus, genus Desulfurococcus, genus Thermoproteus, genus Halobacterium, genus Methanococcus, genus Thermococcus, genus Pyrococcus, genus Methanopyrus, genus Thermoplasma and the like (Lombard J. et al., Mol. Biol. Evol. 2010, 28 (1), 87-99).

In the present invention, the origin of mevalonate pathway as the exogenous NAD(P)H consumption pathway is not particularly limited, but a mevalonate pathway of yeast, prokaryote, or actinomycete is preferable, and a mevalonate pathway of actinomycete is particularly preferable.

Note here that the HMG-CoA reductase includes two types of reductase, i.e., NADPH-dependent HMG-CoA reductase (EC1.1.1.34) and NADH-dependent HMG-CoA reductase (EC1.1.1.88). The HMG-CoA reductase of actinomycete is NADPH-dependent. However, in the present invention, according to the purposes, NADH-dependent HMG-CoA reductase may be used. Examples of the NADH-dependent HMG-CoA reductase include mvaA (P13702) derived from Pseudomonas mevalonii, hmgA-1Mtc_0274 (H8I942) derived from Methanocella conradii, mvaA LLKF_1694 (D2BKK7) derived from Lactococcus lactis subsp. lactis (strain KF147), mvaA SSA_0337 (A3CKT9) derived from Streptococcus sanguinis (strain SK36), or the like.

In the recombinant cell of the present invention, the expression of at least one endogenous NAD(P)H consumption pathway that is different from the exogenous NAD(P)H consumption pathway is down-regulated. Hereinafter, an embodiment of the recombinant cell that is a Clostridium bacterium or a Moorella bacterium, in which the exogenous NAD(P)H consumption pathway is a mevalonate pathway, is specifically described.

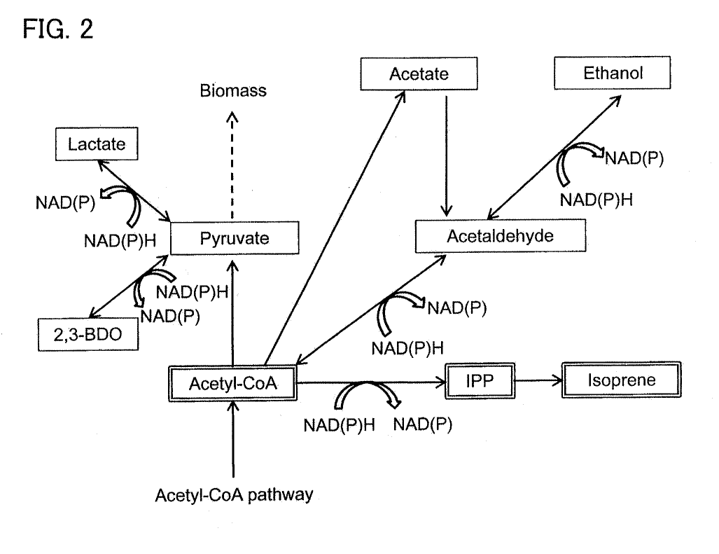

FIG. 2 shows a part of a central metabolic pathway in the Clostridium bacterium or Moorella bacterium in which the mevalonate pathway has been introduced. In the example shown in FIG. 2, IPP is synthesized from acetyl-CoA via the mevalonate pathway (exogenous NAD(P)H consumption pathway). Note here that the acetyl-CoA is supplied from the acetyl-CoA pathway (Wood-Ljungdahl pathway) (FIG. 1).

In an example shown in FIG. 2, the endogenous NAD(P)H consumption pathway includes pathways for carrying out conversion from acetaldehyde to ethanol, conversion from acetyl-CoA to acetaldehyde, conversion from pyruvate to lactate, and conversion from pyruvic acid to 2,3-butanediol, respectively. Herein, the conversion from acetaldehyde to ethanol proceeds by ethanol dehydrogenase, the conversion from acetyl-CoA to acetaldehyde proceeds by acetaldehyde dehydrogenase, the conversion from pyruvic acid to lactate proceeds by lactate dehydrogenase, and the conversion from pyruvate to 2,3-butanediol proceeds by 2,3-butanediol dehydrogenase, respectively.

In this embodiment, expression of at least one of the enzymes involved in the endogenous NAD(P)H consumption pathway is down-regulated. Therefore, an consumption amount of the NAD(P)H in the endogenous NAD(P)H consumption pathway is suppressed, and NAD(P)H is preferentially supplied to the mevalonate pathway that is the exogenous NAD(P)H consumption pathway. As a result, IPP synthesis via the mevalonate pathway is carried out efficiently.

The number of the above-mentioned down-regulated enzyme may be one or more than one.

As mentioned above, the down-regulation of gene expression means regulation for reducing an expression amount of a gene, and regulation for impairing a function of a gene. Examples of the down-regulation of gene expression include a gene knockout and a gene knockdown. Furthermore, the gene knockout includes deletion of the gene itself. Furthermore, an embodiment in which expression amount of a gene is reduced by modifying a gene expression-regulating region of a promoter and the like is also included in the down-regulation of gene expression. When a plurality of down-regulated genes exist, embodiments of the down-regulations of gene may be the same as each other or different from each other.

Examples of a gene knockout method of a Clostridium bacterium or a Moorella bacterium include a technique using a homologous recombination (Leang C. et al., Appl Environ Microbiol. 2013 79(4), 1102-9), a technique using a group II intron (John T. Heap et al., Journal of Microbiological Methods 70 (2007) 452-464), a technique using Cre-Lox66/lox71 (Vel Berzin et al., Appl Bioichem Biotechnol., 2012 168: 1384-1393), and the like.

Note here that an example shown in FIG. 2 includes a pathway in which a conversion from acetyl-CoA to acetic acid is carried out exists other than the NAD(P)H consumption pathway. This pathway proceeds by phosphotransacetylase. Then, in addition to the above-mentioned endogenous NAD(P)H consumption pathway, by down-regulating the expression of phosphotransacetylase, wasteful consumption of acetyl-CoA is suppressed. As a result, the IPP synthesis via the mevalonate pathway is carried out more efficiently.

Furthermore, more preferably, by down-regulating the expression of acetate kinase, generation of excessive hydrocarbon can be suppressed in a pathway in which a conversion from acetyl-CoA to acetic acid is carried out.

An organic compound of not less than C4, which is produced by the recombinant cell of the present invention, is not particularly limited, and examples thereof include terpene. As one embodiment, as a gene that expresses an exogenous NAD(P)H consumption pathway a gene cluster that includes a gene that expresses mevalonic acid and a gene encoding an enzyme for generating isoprene from IPP is employed. Thus, it is possible to provide a recombinant cell capable of producing isoprene.

Examples of the enzyme for producing isoprene from IPP include isoprene synthase. The isoprene synthase is not particularly limited as long as it can exert its enzyme activity in the recombinant cell. Similarly, the gene encoding isoprene synthase is not particularly limited as long as it is normally transcribed and translated in the recombinant cell. The gene encoding isoprene synthase may be codon-modified for ease of transcription in the recombinant cell. For example, when a host cell is a Clostridium bacterium, the codon of the nucleic acid to be introduced may be modified based on the information of codon usage frequency of Clostridium bacteria.

Isoprene synthase is found in many plants. Specific examples of the isoprene synthase include one derived from poplar (Populus nigra) (GenBank Accession No.: AM410988.1). Other examples include one derived from Bacillus subtilis (Sivy T L. et al., Biochem. Biophys. Res. Commu. 2002, 294(1), 71-5).

SEQ ID NO: 1 shows a nucleotide sequence of a gene (DNA) encoding the isoprene synthase derived from poplar and a corresponding amino acid sequence. SEQ ID NO: 2 shows only the amino acid sequence. DNA having the nucleotide sequence of SEQ ID NO: 1 is one example of the nucleic acid encoding isoprene synthase.

Further, the gene encoding isoprene synthase includes at least a nucleic acid encoding the following protein (a), (b) or (c):

(a) a protein consisting of an amino acid sequence of SEQ ID NO: 2,
(b) a protein consisting of an amino acid sequence in which 1 to 20 amino acids are deleted, substituted or added in the amino acid sequence of SEQ ID NO: 2, and having isoprene synthase activity, and
(c) a protein consisting of an amino acid sequence having homology of 60% or more with the amino acid sequence of SEQ ID NO: 2, and having isoprene synthase activity.

The homology of the amino acid sequence in (c) is more preferably 80% or more, further preferably 90% or more, and particularly preferably 95% or more.

As another embodiment, as a gene that expresses an exogenous NAD(P)H consumption pathway, a gene cluster that includes a gene that expresses the mevalonate pathway and a gene encoding an enzyme for producing cyclic terpene from isopentenyl diphosphate is employed. Thus, a recombinant cell capable of producing cyclic terpene can be provided.

Examples of the enzyme for producing cyclic terpene from isopentenyl diphosphate include geranyl diphosphate synthase (GPP synthase) and/or neryl diphosphate synthase (NPP synthase), and cyclic monoterpene synthase.

Any one of the GPP synthase and the NPP synthase may be employed, or both may be employed.

The GPP synthase is not particularly limited as long as it can exert its enzyme activity in the recombinant cell. Similarly, the gene encoding GPP synthase is not particularly limited as long as it is normally transcribed and translated in the recombinant cell.

The same is true for NPP synthase, cyclic monoterpene synthase, and genes encoding synthase.

Specific examples of the GPP synthase include one derived from Arabidopsis thaliana (GenBank Accession No.: Y17376/At2g34630; Bouvier, F., et al., Plant J., 2000, 24, 241-52.), one derived from Mycobacterium tuberculosis (GenBank Accession No.: NP 215504; Mann, F. M., et al., FEBS Lett., 2011, 585, 549-54.), and the like.

SEQ ID NO: 3 shows a nucleotide sequence of the above-mentioned gene (DNA) encoding GPP synthase derived from Arabidopsis thalania and the corresponding amino acid sequence. SEQ ID NO: 4 shows only an amino acid sequence. DNA having the nucleotide sequence of SEQ ID NO: 3 is an example of a gene encoding the GPP synthase.

Furthermore, a gene encoding the GPP synthase includes the nucleic acid encoding protein of the following (a), (b) or (c):

(a) a protein consisting of an amino acid sequence of SEQ ID NO: 4,
(b) a protein consisting of an amino acid sequence in which 1 to 20 amino acids are deleted, substituted or added in the amino acid sequence of SEQ ID NO: 4, and having geranyl diphosphate synthase activity, and
(c) a protein consisting of an amino acid sequence having homology of 60% or more with the amino acid sequence of SEQ ID NO: 4, and having geranyl diphosphate synthase activity.

Note here that the homology of an amino acid sequence in (c) is more preferably 80% or more, further preferably 90% or more, and particularly preferably 95% or more.

Specific examples of the NPP synthase includes one derived from Solanum lycopersicum (GenBank Accession No.: FJ797956), and the like.

SEQ ID NO: 5 shows a nucleotide sequence of the above-mentioned gene (DNA) encoding the NPP synthase derived from Solanum lycopersicum and the corresponding amino acid sequence. SEQ ID NO: 6 shows only an amino acid sequence thereof. DNA having the nucleotide sequence of SEQ ID NO: 5 is an example of a gene encoding the NPP synthase.

Furthermore, a gene encoding the NPP synthase includes at least a nucleic acid encoding protein of the following (d), (e) or (f):

(d) a protein consisting of the amino acid sequence of SEQ ID NO: 6,
(e) a protein consisting of the amino acid sequence in which 1 to 20 amino acids are deleted, substituted or added in the amino acid sequence of SEQ ID NO: 6, and having neryl diphosphate synthase activity, and
(f) a protein consisting of an amino acid sequence having homology of 60% or more with the amino acid sequence of SEQ ID NO: 6, and having neryl diphosphate synthase activity.

Note here that the homology of the amino acid sequence in (f) is more preferably 80% or more, further preferably 90% or more, and particularly preferably 95% or more.

Examples of the cyclic monoterpene synthase include β-phellandrene synthase. By the action of the β-phellandrene synthase, β-phellandrene is synthesized from GPP and/or NPP. Furthermore, as byproduct, 4-carene and limonene can be also synthesized.

Specific examples of the β-phellandrene synthase and genes encoding the same include one derived from Solanum lycopersicum (GenBank Accession No.: FJ797957; Schilmiller, A. L., et al., Proc Natl Acad Sci USA., 2009, 106, 10865-70.), one derived from Lavandula angustifolia (GenBank Accession No.: HQ404305; Demissie, Z. A., et al., Planta, 2011., 233, 685-96), and the like.

SEQ ID NO: 7 shows a nucleotide sequence of the above-mentioned gene (DNA) encoding the β-phellandrene synthase derived from Solanum lycopersicum and the corresponding amino acid sequence. SEQ ID NO: 8 shows only an amino acid sequence.

SEQ ID NO: 9 shows a nucleotide sequence of the above-mentioned gene (DNA) encoding the β-phellandrene synthase derived from Lavandula angustifolia and the corresponding amino acid sequence. SEQ ID NO: 10 shows only an amino acid sequence.

DNA having the nucleotide sequence of SEQ ID NO: 7 or SEQ ID NO: 9 is an example of a gene encoding the β-phellandrene synthase.

Furthermore, a gene encoding the β-phellandrene synthase includes at least a nucleic acid encoding protein of the following (g), (h) or (i):

(g) a protein consisting of the amino acid sequence of SEQ ID NO: 8 or 10;
(h) a protein consisting of the amino acid sequence in which 1 to 20 amino acids are deleted, substituted or added in the amino acid sequence of SEQ ID NO: 8 or 10, and having β-phellandrene synthase activity, and
(i) a protein consisting of an amino acid sequence having homology of 60% or more with the amino acid sequence of SEQ ID NO: 8 or 10, and having β-phellandrene synthase activity.

Note here that the homology of the amino acid sequence in (i) is more preferably 80% or more, further preferably 90% or more, and particularly preferably 95% or more.

Specific embodiments of introducing a gene that expresses an exogenous NAD(P)H consumption pathway and down-regulating the expression of the endogenous NAD(P)H consumption pathway includes substituting a part or whole of an adhE1 gene and an adhE2 gene existing on a genome of a Clostridium bacterium or a Moorella bacterium with the above-mentioned gene cluster by homologous recombination. Both of the adhE1 gene and the adhE2 gene include both of an acetaldehyde dehydrogenase gene and an ethanol dehydrogenase gene, and both are located adjacent to each other on the genome.

The recombinant cell of the present invention includes a gene that expresses an exogenous NAD(P)H consumption pathway. For example, a gene that expresses an exogenous NAD(P)H consumption pathway is introduced into a host cell. The introduced gene may be incorporated into the genome of the host cell, or may exist out of the genome in a state in which it is incorporated in a plasmid.

The method of introducing a gene into the host cell is not particularly limited, and may be selected appropriately depending on the kind of the host cell and the like. For example, a vector that can be introduced into the host cell and can allow expression of the gene incorporated therein may be used.

For example, when the host cell is a prokaryote such as a bacterium, a vector that can self duplicate or can be incorporated in chromosome in the host cell, and contains a promoter at the position allowing transcription of the inserted gene (DNA) can be used. For example, it is preferred to construct in the host cell a series of structures including a promoter, a ribosome binding sequence, the above gene (DNA) and a transcription termination sequence by using the vector.

In the case where the host cell is a Clostridium bacterium (including related species such as Moorella bacteria), a shuttle vector pIMP1 between Clostridium bacterium and Escherichia coli (Mermelstein L D et al., Bio/technology 1992, 10, 190-195) may be used. The shuttle vector is a fusion vector of pUC9 (ATCC 37252) and pIM13 isolated from Bacillus subtilis (Projan S J et al., J. Bacteriol. 1987, 169 (11), 5131-5139) and is retained stably in the Clostridium bacterium. Other examples of a shuttle vector between Clostridium bacterium and E. coli include pSOS95 (GenBank: AY187686.1).

For gene introduction into the Clostridium bacterium, an electroporation method is generally used. However, the introduced exogenous plasmid immediately after gene introduction is liable to be decomposed by a restriction enzyme Cac824I and the like, and is therefore very instable. For this reason, it is preferred to once amplify the vector from pIMP1 in E. coli, for example, strain ER2275 having pAN1 (Mermelstein L D et al., Apply. Environ. Microbiol. 1993, 59(4), 1077-1081) carrying a methyl transferase gene from Bacillus subtilis phage ΦT1, followed by a methylation treatment, and to recover the resultant vector from E. coli for use in transformation by electroporation. Recently, Cac824I gene-deficient Clostridium acetobuthylicum has been developed, and make it possible to stably carry a vector which is not subjected to a methylation treatment (Dong H. et al., PLoS ONE 2010, 5 (2), e9038). Furthermore, it should be noted that an unmethylated vector is efficiently introduced by amplifying the vector with NEB express which is an improved version of E. coli BL21 strain (Leang C. et al., Appl Environ Microbiol. 2013 79(4), 1102-9). Also, it should be noted that a gene is introduced into a genome of a Clostridium bacterium by using pBluescipt II KS(−) or pUC19 vector into which a sequence homologous to that of the host cell is integrated (Leang C. et al., Appl Environ Microbiol. 2013 79(4), 1102-9, Berzin V. et al., Appl Biochem Biotechnol (2012) 167:338-347).

Examples of the promoter for heterologous gene expression in Clostridium bacteria include thl (thiolase) promoter (Perret S et al., J. Bacteriol. 2004, 186(1), 253-257), Dha (glycerol dehydratase) promoter (Raynaud C. et al., PNAS 2003, 100(9), 5010-5015), ptb (phosphotransbutyrylase) promoter (Desai R P et al., Appl. Environ. Microbiol. 1999, 65(3), 936-945), and adc (acetoacetate decarboxylase) promoter (Lee J et al., Appl. Environ. Microbiol. 2012, 78 (5), 1416-1423). However, in the present invention, sequences of promoter regions used in operons of various metabolic systems found in the host cell or the like may be used without limited to the above examples.

Furthermore, promoters that regulate pta (phosphate acetyltransferase), adhE (aldehyde/alcohol dehydrogenase), CODH (carbon monoxide dehydrogenase), acsA (acetyl-coA synthase a subunit), ferredoxin, Rnf complex, hydrogenase, GroE, ATP synthase, or the like can be used. In the case of conducting a syngas fermentation, promoters that can be activated by carbon monoxide, carbon dioxide, or hydrogen are also suitable.

For introducing plural kinds of genes into the host cell by using a vector, the genes may be incorporated in one vector, or incorporated in individual vectors. When plural kinds of genes are incorporated in one vector, these genes may be expressed under a common promoter for these genes, or expressed under individual promoters. An exemplary form of introducing plural kinds of genes include a mode of introducing isoprene synthase gene, GPP synthase gene, NPP synthase gene, cyclic monoterpene synthase gene, or the like in addition to the gene acting in the mevalonate pathway.

As described above, while the known vectors that can be used in the present invention have been shown, the region involved in transcription control and replication regions such as promoter and terminator can be modified depending on the purpose. The modification includes change to other natural gene sequence in each host cell or its related species, and change to an artificial gene sequence.

One aspect of the method for producing an organic compound of the present invention includes bringing at least one C1 compound selected from the group consisting of carbon monoxide and carbon dioxide into contact with the above-mentioned recombinant cell to produce an organic compound having 4 or more carbon atoms from the C1 compound in the recombinant cell.

In a preferable embodiment, the recombinant cell is cultured by using at least one C1 compound selected from the group consisting of carbon monoxide and carbon dioxide as a carbon source to produce an organic compound having 4 or more carbon atoms in the recombinant cell. The C1 compound used as a carbon source may be used singly or in combination of two or more. Furthermore, the C1 compound is preferably used as a main carbon source, and more preferably as the only carbon source.

Furthermore, it is preferable to concurrently provide hydrogen (H₂) as an energy source.

The method for culturing the recombinant cell of the present invention is not particularly limited, and can be appropriately carried out depending on the type of the recombinant cell, and the like. When the recombinant cell is a Clostridium bacterium (obligatory anaerobic and strictly anaerobic), it is cultured, for example, in a nutrient condition including inorganic salts required for growth, and syngas. Preferably, it is cultured under a pressurized condition at about 0.2 to 0.3 MPa (absolute pressure). Furthermore, for improving initial proliferation and attained cell density, small amounts of organic substances such as vitamins, yeast extract, corn steep liquor, and Bacto Tryptone, may be added.

Note here that the recombinant cell is aerobic or facultative anaerobic, for example, it may be cultured in a liquid medium under aeration and stirring.

Methods other than culture can be employed. That is, the organic compound can be produced by bringing the above-mentioned C1 compound into contact with the recombinant cell regardless of whether or not cell division (cell proliferation) occurs. For example, the above-mentioned C1 compound is continuously fed to the immobilized recombinant cell, so that the organic compound can be continuously produced. Also in this case, the C1 compound used may be used singly or in combination of two or more. Furthermore, it is preferable to bring hydrogen (H₂) into contact concurrently as an energy source.

In a preferable embodiment, the recombinant cell is provided with a gas mainly containing carbon monoxide, a gas mainly containing carbon monoxide and hydrogen, a gas mainly containing carbon dioxide and hydrogen, or a gas mainly containing carbon monoxide, carbon dioxide and hydrogen. In other words, the organic compound is produced from carbon monoxide or carbon dioxide in such a gas by culturing the recombinant cell by using the above-mentioned gas as a carbon source, or by bringing the above-mentioned gas into contact with the recombinant cell. Also in this case, hydrogen is used as an energy source.

In addition to these C1 compounds, the recombinant cell may be provided with formic acid or methanol. In other words, the recombinant cell is cultured by using formic acid or methanol and the above-mentioned gas is used as a carbon source, or formic acid or methanol and the above-mentioned gas are brought into contact with the recombinant cell. Addition of formic acid or methanol allows for the culture efficiency and the production efficiency of the organic compound to be improved. Examples of the embodiment of providing include concurrently providing a recombinant cell with formic acid or methanol and the above-mentioned gas.

According to the embodiment in which a gene cluster that includes a gene that expresses a mevalonate pathway and a gene encoding an enzyme for producing isoprene from isopentenyl diphosphate as the gene expressing an exogenous NADP consumption pathway, isoprene can be produced as the organic compound.

Furthermore, according to the embodiment in which a gene cluster including a gene that expresses a mevalonate pathway and a gene encoding an enzyme for producing cyclic terpene from isopentenyl diphosphate, as the gene expressing an exogenous NADP consumption pathway, cyclic terpene can be produced as the organic compound. According to an embodiment in which the enzyme for producing cyclic terpene from isopentenyl diphosphate is geranyl diphosphate synthase and/or neryl diphosphate synthase, as well as cyclic monoterpene synthase, cyclic monoterpene can be produced. Furthermore, according to an embodiment in which the cyclic monoterpene synthase is β-phellandrene synthase, as the cyclic monoterpene, β-phellandrene, 4-carene, or limonene can be produced.

The produced organic compound is accumulated in the cell or released to the outside of the cell. For example, in an embodiment in which cyclic terpene is produced, by using the recombinant cell prepared from a host cell of a Clostridium bacterium or a Moorella bacterium, and collecting cyclic monoterpene that has been released to the outside of the cell, followed by performing isolation and purification by, for example, distillation, purified cyclic terpene can be obtained.

Furthermore, methods for isolation and purification of the organic compound from the cultured product of the recombinant cell are not particularly limited. When cyclic terpene is produced, for example, a culture solution (a culture supernatant) can be extracted with an appropriate solvent such as pentane, and purified to a high purity by chromatography such as reverse-phase chromatography and gas chromatography. Since most of cyclic terpene released to the outside of the cell is evaporated into a vapor phase, it may be liquefied and collected by a cold trap and the like.

Bicarbonate can be sometimes used in place of carbon dioxide. In other words, Clostridium bacteria and related species are known to have carbonic anhydrase (CA) (EC 4.2.1.1: H₂O+CO₂⇔HCO₃⁻+H⁺) (Braus-Stromeyer S A et al., J. Bacteriol. 1997, 179(22), 7197-7200). Bicarbonate such as NaHCO₃ which is a source of HCO₃⁻ can be used as a CO₂ source.

Herein, combinations of carbon monoxide, carbon dioxide, formic acid, and methanol that can be provided to the recombinant cell in the case where the host cell has the acetyl-CoA pathway and the methanol pathway (FIG. 1) are described.

In acetyl-CoA synthesis by the acetyl-CoA pathway, a synthesis process of acetyl-CoA from CoA, methyltetrahydrofolate ([CH₃]-THF), and CO by the actions of methyltransferase, Corrinoid iron-sulfur protein (CoFeS—P), acetyl-CoA synthase (ACS), and CODH is essential (Ragsdale S W et al., B.B.R.C. 2008, 1784(12), 1873-1898).

On the other hand, it is known that adding formic acid and/or methanol besides CO and/or CO₂ as a carbon source in culturing of Butyribacterium methylotrophicum increases the content of tetrahydrofolate (THF) in CO metabolism, namely, methyl branch in the acetyl-CoA pathway, and activities of CODH, formate dehydrogenase (FDH) and hydrogenase required in CO metabolism (Kerby R. et al., J. Bacteriol. 1987, 169(12), 5605-5609). Also in Eubacterium limosum or the like, it is demonstrated that high proliferation is achieved by using CO₂ and methanol as a carbon source in an anaerobic condition (Genthner B R S. et al., Appl. Environ. Microbiol., 1987, 53(3), 471-476).

The influence of methanol on syngas utilizing microorganisms, and the results of genome analysis of Moorella thermoacetica (Clostridium thermoaceticum), Clostridium ljungdahlii and the like (Pierce E. et al., Environ. Microbiol. 2008, 10(10), 2550-2573; Dune P. et al., PNAS 2010, 107(29), 13087-13092) can give an explanation for involvement of the methanol pathway as shown in FIG. 1 as a donor of a methyl group in the acetyl-CoA pathway (Wood-Ljungdahl pathway) in these microorganism species.

Actually in some Clostridium bacteria, the forward activity of formate dehydrogenase (FDH) (EC 1.2.1.2/1.2.1.43: Formate+NAD(P)⁺⇔CO₂+NAD(P)H) (formation of CO₂ from formate) is confirmed (Liu C L et al., J. Bacteriol. 1984, 159(1), 375-380; Keamy J J et al., J. Bacteriol. 1972, 109(1), 152-161). Therefore, in these strains, a reaction in the direction of generating CO₂ from methanol (CH₃OH) and/or formic acid (HCOOH) can partly proceed when CO₂ and/or CO is deficient (FIG. 1). This can also be explained by the phenomenon that formate dehydrogenase activity and CODH activity increase by addition of CH₃OH (Kerby R et al., J. Bateriol. 1987, 169(12), 5605-5609) as described above. In other words, these can be proliferated with formic acid (HCOOH) or methanol (CH₃OH) as the sole carbon source.

Even if the host cell strain inherently lacks the forward activity of formate dehydrogenase, it may be provided with the forward activity by gene modification such as introduction of mutation, introduction of foreign gene, or genome shuffling.

For these reasons, it is possible to produce the aforementioned organic compound using the following gas or liquid when the host cell has the acetyl-CoA pathway and the methanol pathway.

CO

CO₂

CO/H₂

CO₂/H₂

CO/CO₂/H₂

CO/HCOOH

CO₂/HCOOH

CO/CH₃OH

CO₂/CH₃OH

CO/H₂/HCOOH

CO₂/H₂/HCOOH

CO/H₂/CH₃OH

CO₂/H₂/CH₃OH

CO/CO₂/H₂/HCOOH

CO/CO₂/H₂/CH₃OH

CH₃OH/H₂

HCOOH/H₂

CH₃OH

HCOOH

When the recombinant cell of the present invention is cultured exclusively for cell proliferation, rather than for production of the organic compound, it is not necessary to use carbon monoxide and/or carbon dioxide as a carbon source. For example, the recombinant cell may be cultured using other carbon sources such as saccharides or glycerin which is easily assimilated by the recombinant cell of the present invention

In the following, the present invention will be described more specifically by way of Examples. However, the present invention is not limited to these examples.

### Example 1

In this Example, in the recombinant cell of Clostridium ljungdahlii as one type of syngas assimilating bacteria, high efficiency production of β-phellandrene was carried out.

(1) Construction of Various Vectors

With reference to Appl Biochem Biotechnol (2012) 168:1384-1393 and Bioprocess Biosyst Eng (2014) 37:245_260, pUC-ΔadhE-Cat (SEQ ID NO: 11) including an adhE1 (CLJU_c16510) upstream sequence of C. ljungdahlii, a lox66 sequence, a chloramphenicol resistant gene (FM201786), a lox71 sequence, and an adhE2 (CLJU_c16520) downstream sequence of C. ljungdahlii was prepared.

Furthermore, pJIR750ai (Sigma-Aldrich) that is a Clostridium/E. coli binary vector was modified to construct pSK1-PHS-NPPS (SEQ ID NO: 12) including a nucleotide sequence in which a β-phellandrene synthase gene (GenBank Accession No.: FJ797957; Schilmiller, A. L., et al., Proc Natl Acad Sci USA., 2009, 106, 10865-70.), a neryl diphosphate (NPP) synthase gene (GenBank Accession No.: FJ797956), and a chloramphenicol resistant gene (FM201786) had been codon-modified.

Furthermore, in addition to the sequence of SEQ ID NO: 12, pSK1-PHS-NPPS-MVA (SEQ ID NO: 13) further including a mevalonate pathway gene cluster derived from actinomycete (including mvaA derived from Pseudomonas mevalonii) was constructed. Each sequence is codon-modified with consideration of the codon usage frequency of a Clostridium bacterium.

(2) Preparation of adhE Gene Knock Out Clostridium Strain

pUC-ΔadhE-Cat was introduced into C. ljungdahlii (DSM13528/ATCC55383) using a technique recommended in Leang C. et al., Appl Environ Microbiol. 2013 79(4), 1102-9, and selected in an ATCC1754 agar medium (1.5% agar) including 10 μg/mL chloramphenicol to produce adhE1/adhE2 knock out strains (ΔadhE strains). A ΔadhE strain competent cell was prepared using a technique recommended in Leang C. et al., Appl Environ Microbiol. 2013 79(4), 1102-9. A pUCΔadhE strain competent cell was subjected to Cre-recombinase treatment to remove a chloramphenicol resistant gene by a technique described in Bioprocess Biosyst Eng (2014) 37: 245-260 so as to obtain a chloramphenicol sensitive strain (ΔadhEΔCm strain).

(3) Gene Introduction into DSM13528/ATCC55383 Strain and ΔadhEΔCm Strain

The pSK1-PHS-NPPS and the pSK1-PHS-NPPS-MVA were introduced into the DSM13528/ATCC55383 strain, and the pSK1-PHS-NPPS-MVA was introduced into the ΔadhEΔCm strain, respectively, by electroporation using a technique described in Leang C. et al., Appl Environ Microbiol. 2013 79(4), 1102-9, and selected in an ATCC1754 agar medium containing 10 μg/mL chloramphenicol (1.5% agar containing fructose).

(4) β-Phellandrene Quantification

The pSK1-PHS-NPPS-introduced DSM13528/ATCC55383 strain, the pSK1-PHS-NPPS-MVA-introduced DSM13528/ATCC55383 strain, and the pSK1-PHS-NPPS-MVA-introduced ΔadhEΔCm strain selected in the above item (3) were respectively cultured at 37° C. in an aerobic condition. As a culture medium for inoculation, 5 mL of an ATCC1754 medium containing 10 μg/mL chloramphenicol (pH=5.0, fructose was not contained) was used. A 27 mL-volume hermetically-sealable headspace vial vessel was charged with a mixed gas of CO/CO₂/H₂=33/33/34% (volume ratio), and filled with the mixed gas at a gas pressure of 0.25 MPa (absolute pressure), and hermetically sealed with an aluminum cap, and then shaking culture was conducted. In the cultured product in which proliferation was found, at the time when OD600 reached 1.0, culture was terminated, and the vapor phase was analyzed using a gas chromatograph mass spectrometer (Shimadzu GCMS-QP2010 Ultra).

As a result, in the pSK1-PHS-NPPS-introduced DSM13528/ATCC55383 strain, β-phellandrene was detected in a production amount of 0.15 mg on average per dry cell (g). In the pSK1-PHS-NPPS-MVA-introduced DSM13528/ATCC55383 strain, β-phellandrene was detected in a production amount of 10 mg on average per dry cell (g). In the pSK1-PHS-NPPS-MVA-introduced ΔadhEΔCm strain, β-phellandrene was detected in an amount of 55 mg on average per dry cell (g).

As mentioned above, it was shown that when a foreign mevalonate pathway for producing a β-phellandrene precursor was introduced into a host cell, the production amount of the β-phellandrene that is one type of cyclic monoterpene was found to be increased. In addition, it was shown that when adhE1 and adhE2 genes for the NAD(P)H consumption pathway were knocked out, β-phellandrene can be produced more efficiently.

### Example 2

In this Example, a sequence in which a gene cluster that includes a genome sequence of C. ljungdahlii, as well as an exogenous mevalonate pathway and isoprene synthase had been introduced into pUC57 (GenBank Accession No.Y14837) was constructed, and introduced into C. ljungdahlii by homologous recombination to prepare a recombinant cell in which adhE1 and adhE2 of the host cell had been knocked out, and the gene cluster had been introduced into a genome. Furthermore, isoprene was produced in this recombinant cell at high efficiency.

(1) Construction of Various Vectors

A gene cluster was introduced into pUC57 (GenBank Accession No. Y14837) to construct pUC-ΔadhE-IspS-MVA (SEQ ID NO: 14), the gene cluster including an adhE1 (CLJU_c16510) upstream sequence and an adhE2 (CLJU_c16520) downstream sequence both of C. ljungdahlii (DSM13528/ATCC55383), a mevalonate pathway gene derived from actinomycete (including mvaA derived from Pseudomonas mevalonii), an isoprene synthase gene derived from Populus nigra (GenBank Accession No.: AM410988.1), and a chloramphenicol resistant gene (FM201786) (Appl Biochem Biotechnol. 2012 May; 167(2):338-47.). Furthermore, three genes were introduced into the sequence-modified pJIR750ai (Sigma-Aldrich) to construct pSK1-IspS-MVA (SEQ ID NO: 15), the three genes including an isoprene synthase gene derived from Populus nigra (GenBank Accession No.: AM410988.1), a chloramphenicol resistant gene (FM201786), and a mevalonate pathway gene derived from actinomycete (including mvaA derived from Pseudomonas mevalonii). Each sequence was codon-modified with consideration of the codon usage frequency of a Clostridium bacterium.

(2) Gene Introduction

The pUC-ΔadhE-IspS-MVA was introduced into C. ljungdahlii (DSM13528/ATCC55383) by the electroporation technique of Leang C. et al. and selected in an ATCC1754 agar medium containing 10 μg/mL chloramphenicol (1.5% agar containing fructose). A reconbinant cell in which deletion of adhE1 and adhE2 and introduction of an isoprene synthase gene into a genome by gene introduction of pUC-ΔadhE-IspS-MVA had been recognized was selected and referred to as a ΔadhE-IspS-MVA strain. Furthermore, pSK1-IspS-MVA was introduced into C. ljungdahlii (DSM13528/ATCC55383) and ΔadhEΔCm strain prepared in Example 1, respectively, by the electroporation technique of Leang C. et al, followed by selecting in an ATCC1754 agar medium containing 10 μg/mL chloramphenicol (1.5% agar containing fructose).

(3) Isoprene Quantification

The ΔadhE-IspS-MVA strain, pSK1-IspS-MVA-introduced DSM13528/ATCC55383, pSK1-IspS-MVA-introduced ΔadhEΔCm strain that had been obtained in the above item (2) were respectively cultured at 37° C. in an anaerobic condition. A 27 mL-volume hermetically-sealable headspace vial vessel was charged with 5 mL of ATCC medium 1754 PETC medium containing 10 μg/mL chloramphenicol (pH=5.0, fructose was not contained), and filled with a mixed gas of CO/CO₂/H₂=33/33/34% (volume ratio) at a gas pressure of 0.25 MPa (absolute pressure), and sealed with an aluminum cap, and then shaking culture was conducted. The culture was terminated at the time when OD600 reached 1.0, and the vapor phase was analyzed using a gas chromatograph mass spectrometer (Shimadzu GCMS-QP2010 Ultra). As a result, in the ΔadhE-IspS-MVA strain, 185 mg on average of isoprene per dry cell (g) was detected. In the pSK1-IspS-MVA-introduced ΔadhEΔCm strain, 74 mg on average of isoprene per dry cell (g) was detected. In pSK1-IspS-MVA-introdued DSM13528/ATCC55383, 15 mg of isoprene on average per dry cell (g) was detected.

As mentioned above, it was shown that when a foreign mevalonate pathway for producing an isoprene precursor was introduced into a host cell, and adhE1 and adhE2 genes for the host NAD(P)H consumption pathway were knocked out, isoprene can be produced more efficiently. Furthermore, it was shown that by incorporating the nucleotide sequence into a genome by homologous recombination, isoprene could be produced more efficiently.

1. A recombinant cell having a function of synthesizing acetyl-CoA from methyltetrahydrofolate, carbon monoxide, and CoA, comprising:
a gene that expresses an exogenous NAD(P)H consumption pathway, the gene being expressed in the recombinant cell,
wherein expression in at least one of endogenous NAD(P)H consumption pathways of the recombinant cell is down-regulated, and the endogenous NAD(P)H consumption pathway is different from the exogenous NAD(P)H consumption pathway, and
the recombinant cell produces an organic compound having 4 or more carbon atoms from at least one selected from the group consisting of carbon monoxide and carbon dioxide via the exogenous NAD(P)H consumption pathway.

2. The recombinant cell according to claim 1, being a Clostridium bacterium or a Moorella bacterium.

3. (canceled)

4. The recombinant cell according to claim 2, wherein the exogenous NAD(P)H consumption pathway is a mevalonate pathway.

5. The recombinant cell according to claim 4, wherein the mevalonate pathway is a mevalonate pathway of yeast, prokaryote, or actinomycete.

6. (canceled)

7. The recombinant cell according to claim 4, wherein HMG-CoA reductase of the mevalonate pathway includes NADH-dependent HMG-CoA reductase.

8. (canceled)

9. The recombinant cell according to claim 4, wherein in the endogenous NAD(P)H consumption pathway, expression of at least one selected from the group consisting of ethanol dehydrogenase, acetaldehyde dehydrogenase, lactate dehydrogenase, and 2,3-butanediol dehydrogenase is down-regulated.

10. The recombinant cell according to claim 9, wherein in the endogenous NAD(P)H consumption pathway, expression of at least ethanol dehydrogenase is down-regulated.

11. The recombinant cell according to claim 10, wherein in the endogenous NAD(P)H consumption pathway, expression of acetaldehyde dehydrogenase is further down-regulated.

12. (canceled)

13. The recombinant cell according to claim 9, wherein at least one of the down-regulation is a lack of expression.

14. The recombinant cell according to claim 13, wherein expression of ethanol dehydrogenase and/or acetaldehyde dehydrogenase lacks.

15. The recombinant cell according to claim 14, wherein instead of a gene encoding ethanol dehydrogenase and/or acetaldehyde dehydrogenase, a gene that expresses the mevalonate pathway is incorporated in a genome by homologous recombination.

16-18. (canceled)

19. The recombinant cell according to claim 9, wherein the gene that expresses the exogenous NAD(P)H consumption pathway is a gene cluster that includes a gene that expresses a mevalonate pathway and a gene encoding an enzyme for producing isoprene from isopentenyl diphosphate, and the organic compound is isoprene.

20. The recombinant cell according to claim 19, wherein the enzyme for producing isoprene from isopentenyl diphosphate is isoprene synthase.

21-24. (canceled)

25. The recombinant cell according to claim 19, wherein a part or whole of an adhE1 gene and an adhE2 gene of a host cell that is a basis of the recombinant cell is deleted, and the gene cluster, instead of the adhE1 gene and the adhE2 gene, is incorporated in a genome by homologous recombination.

26. The recombinant cell according to claim 21, wherein a part or whole of an adhE1 gene and an adhE2 gene of a host cell that is a basis of the recombinant cell is deleted, and the gene cluster, instead of the adhE1 gene and the adhE2 gene, is incorporated in a genome by homologous recombination.

27. A method for manufacturing a recombinant cell, the method comprising:
a first step of providing a host cell having a function of synthesizing acetyl-CoA from methyltetrahydrofolate, carbon monoxide, and CoA; and
a second step of introducing a gene that expresses an exogenous NAD(P)H consumption pathway into the host cell,
wherein
the gene that has been introduced in the second step is expressed in the host cell;
the expression in at least one of endogenous NAD(P)H consumption pathways of the host cell is down-regulated, and the endogenous NAD(P)H consumption pathway is different from the exogenous NAD(P)H consumption pathway; and
the recombinant cell produces the organic compound having 4 or more carbon atoms from at least one selected from the group consisting of carbon monoxide and carbon dioxide, via the exogenous NAD(P)H consumption pathway.

28-52. (canceled)

53. A method for producing an organic compound, the method comprising: bringing at least one C1 compound selected from the group consisting of carbon monoxide and carbon dioxide into contact with the recombinant cell according to claim 1.

54. The method according to claim 53, wherein the recombinant cell is cultured using at least one C1 compound selected from the group consisting of carbon monoxide and carbon dioxide as a carbon source to produce an organic compound having 4 or more carbon atoms in the recombinant cell.

55. The method according to claim 53, wherein the recombinant cell is provided with gas mainly containing carbon monoxide, gas mainly containing carbon monoxide and hydrogen, gas mainly containing carbon dioxide and hydrogen, or gas mainly containing carbon monoxide, carbon dioxide and hydrogen.

56. (canceled)

57. The method according to claim 53, wherein the recombinant cell is a Clostridium bacterium or a Moorella bacterium.

58-59. (canceled)
